SEQUENCE LISTING (9 sequences)

SEQ ID NO: 1 - protein, 9 aa, human
ILDSSEEDK

SEQ ID NO: 2 - protein, 9 aa, human
YADQVNIDY

SEQ ID NO: 3 - protein, 9 aa, human
ATEKSVKTK

SEQ ID NO: 4 - protein, 9 aa, human
FTEEIEWLK

SEQ ID NO: 5 - protein, 9 aa, human
DQMEEEEFI

SEQ ID NO: 6 - protein, 9 aa, human
QLFEDNYAL

SEQ ID NO: 7 - protein, 9 aa, human
FLVFVFVFL

SEQ ID NO: 8 - protein, 9 aa, human
LMWITQCFL

SEQ ID NO: 9 - protein, 9 aa, human
TLAKFSPYL

(19) United States
(12) Patent Application Publication
(10) Pub. No.: US 2015/0335679 A1
(43) Pub. Date: Nov. 26, 2015

(54) Methods and Compositions for Treating Malignancies with Dendritic Cells

(71) Applicant: Kiromic, LLC, Lubbock, TX (US)

(72) Inventor: Maurizio Chiriva-Internati, Lubbock, TX (US)

(21) Appl. No.: 14/717,090

(22) Filed: May 20, 2015

Related U.S. Application Data
(60) Provisional application No. 62/000,608, filed on May 20, 2014.

(51) Int. Cl. A61K 35/15

(52) U.S. Cl. CPC A61K 35/15

(57) ABSTRACT

This invention provides methods and compositions for the treatment and/or prevention of cancer, including solid malignancies and hematologic malignancies. In particular, the present invention relates to immunotherapy using antigen-presenting cells loaded with tumor associated peptide antigens (TAPAs). If a subject expresses at least one tumor-associated antigen, the subject can be treated by antigen-presenting cells (e.g., dendritic cells) loaded with at least one tumor-associated peptide antigen derived from the tumor-associated antigen (s) expressed by the subject. This personalized immunotherapy induces or enhances immune responses to cells that express tumor-associated antigen(s).

### CROSS REFERENCE TO RELATED APPLICATION

This application claims priority to U.S. Provisional Application No. 62/000,608 filed on May 20, 2014, which is incorporated herein by reference in its entirety.

### FIELD OF THE INVENTION

The present invention relates to methods and compositions for the treatment and/or prevention of cancer. In particular, the present invention relates to immunotherapy using antigen-presenting cells loaded with Tumor Associated Peptide Antigens (TAPAs).

### BACKGROUND OF THE INVENTION

Despite advances in understanding the biology of solid malignancies (SM), and the availability of new treatment options, the vast majority of patients with SM remain incurable [1]. This is especially true for elderly patients and those whose disease proves refractory and/or relapses following standard therapy. Therefore, therapeutic options for this patient population are limited and novel strategies are needed for patients with progressive and/or refractory SM.

Immunotherapy has recently emerged as a promising treatment strategy for patients with SM [2]. Dendritic cells (DCs) are potent antigen-presenting cells (APCs) and are important for the efficient priming and activation of naïve T-cells against specific antigens. DCs undergo maturation and activation in the presence of specific immunogenic antigens and serve as a critical link between innate and adaptive immunity resulting in antitumor responses [3]. It is now possible to generate human DCs in vitro from monocytes exposed to GM-CSF and IL-4, and DC-based treatments have become an attractive immunotherapeutic strategies for cancer in the last decade. DC vaccination studies have been conducted in several different neoplasms [4,5] with variable results [52].

Since the majority of patients with metastatic or progressive SM display a defective immune response to tumor antigens, the ex vivo activation of DCs, through their exposure to tumor associated antigens, is an attractive and active area of investigation [6,7]. A variety of early clinical studies have used antigen-pulsed DC therapy in patients with SM malignancies including metastatic prostate, hepatocellular, colorectal, renal, cervical, and breast carcinomas, as well as melanomas and glioblastomas [8-13]. The strategies tested have included pulsing DCs with whole tumor cell lysates or specific tumor-associated antigens [8-13]. Although safe, DC vaccination strategies have resulted in unsatisfactory clinical outcomes attributed to either the poor immunogenicity of the target antigens, suboptimal DC-dosing, generation of tolerogenic DC subtypes and activity of suppressor regulatory T-cells (Treg).

DCs can be generated from blood-derived monocytes in the presence of GM-CSF and IL-4 and administered to cancer patients [14, 15]. Moreover, DCs pulsed with specific Tumor Associated Peptide Antigens (TAPAs) are capable of eliciting immune and antitumor responses, without prohibitive toxicity [8-13, 16]. Despite these advances, many questions remain on how to improve this immunotherapeutic strategy, including how to best activate DCs with various antigens and cytokine combinations, the characterization of “ideal” TAPAs capable of eliciting strong and durable immune responses in patients with SM, effective vaccine immune adjuvants, the optimal dosing and schedule of DC treatments, how to inhibit inhibitory signals from Tregs and what specific group of patients with SM are best suited for this particular DC-based vaccination approach. To solve these problems, several potential strategies have been proposed, including the use of more potent and immunogenic TAPAs and/or Th1 polarizing cytokines [18, 19], enhancement ex vivo DC maturation and activation through Toll-like receptor (TLR) signaling [20], cross presentation of neoplastic cell-associated antigens [21], stimulation of natural killer cell and/or T-helper activity to generate Th1 polarization in DCs [22], inhibition of immunosuppressive Treg cell populations [23] and regulation of DC migration to regional lymph nodes [24], among others. However, there still remain the need to optimize DC-based immunotherapy to improve responses in cancer patients.

### SUMMARY

The present application provides for a method of treating and/or preventing cancer in a subject. The method may comprise the following steps: (a) determining whether the subject expresses at least one tumor-associated antigen; (b) loading antigen-presenting cells with at least one tumor-associated peptide antigen (TAPA) derived from at least one tumor-associated antigen expressed by the subject; and (c) administering the antigen-presenting cells from step (b) to the subject.

Prior to administration of tumor-associated peptide antigen-loaded antigen presenting cells to the subject (e.g., before step (c) the method), at least one immunosuppressive agent may be administered to the subject. In certain embodiments, the immunosuppressive agent may be an alkylating agent. In one embodiment, the alkylating agent is cyclophosphamide.

After administration of tumor-associated peptide antigen-loaded antigen presenting cells to the subject (e.g., after step (c) the method), granulocyte-macrophage colony-stimulating factor (GM-CSF) may be administered to the subject.

To determine whether the subject expresses a tumor-associated antigen, the protein level and/or mRNA level of at least one tumor-associated antigen can be assayed, for example, by RT-PCR, Western blot, immunohistochemistry, enzyme-linked immunosorbent assay (ELISA), or combinations thereof.

The antigen-presenting cells may be dendritic cells, macrophages, B cells, etc. The dendritic cells may be derived from autologous monocytes. For example, the monocytes can be cultured in vitro to induce differentiation into dendritic cells. In one embodiment, the differentiation into dendritic cells is facilitated by at least one maturation factor, including, but not limited to, IL-1β, TNFα, IFN-α, poly (I:C) and a combination thereof. The monocytes may be isolated from the subject's blood. Before the subject's blood is obtained, granulocyte-macrophage colony-stimulating factor (GM-CSF) may be administered to the subject.

The cancer may be solid malignancy or hematologic malignancy. The subject or the cancer cells may express at least one tumor-associated antigen. In one embodiment, the subject has metastatic solid malignancy. In another embodiment, the subject has progressive and/or refractory solid malignancy.

Also encompassed by the present application is a pharmaceutical composition comprising dendritic cells loaded with at least one tumor-associated peptide antigen (TAPA).

The present application provides for a pharmaceutical composition comprising dendritic cells comprising nucleic acids encoding at least one tumor-associated antigen.

In one embodiment, the tumor-associated antigen comprises (or consists essentially of, or consists of) Sp17, Ropporin, AKAP-4, PTTG1, Span-xb, Her-2/neu, HM1.24, NY-ESO-1, MAGE-1 or combinations thereof. In one embodiment, the TAPA comprises (or consists essentially of, or consists of) SEQ ID NOs: 1, 2, 3, 4, 5, 6, 7, 8, or combinations thereof. The TAPA may be MHC class I-restricted or MHC class II-restricted. For example, The MHC class I may be HLA-A, HLA-B, HLA-C, HLA-E, HLA-F, HLA-G, HLA-K, or HLA-L.

### BRIEF DESCRIPTION OF THE FIGURES

FIG. 1 shows total number of PBMCs isolated from 35 mL of peripheral blood, total non-adherent cells removed, and viability to cells after Ficoll purification and after 3-h adhesion.

FIG. 2 shows representative phenotypic characterization of DC after 2 or 5 days of GM-CSF stimulation, and after maturation/SP17(103-111) peptide (SEQ ID NO. 1) pulse.

FIG. 3 shows results of the effects of GM-CSF and IL-4 and of the maturation/pulsing stimulation on the phenotype of monocyte-derived DC.

FIG. 4 shows representative microphotographs (20× magnification) of PBL-DC co-cultures showing the formation of rosetta structures, indicating positive DC-T cell interactions.

FIG. 5 shows representative results of cytotoxicity assays obtained with DC pulsed with SP17(103-111) peptide (SEQ ID NO. 1), AKAP-4(150-158) peptide (SEQ ID NO. 2), PTTG1(70-78) peptide (SEQ ID NO. 3), Ropporin-1(113-121) peptide (SEQ ID NO. 4), or a mixture of these four antigens. Results demonstrate that the DC generated with the protocol described here successfully induced the maturation and activation of antigen-specific cytotoxic lymphocytes. Lack of significant lysis of targets expressing the irrelevant antigen, E6, confirmed target specificity of the DC-primed CTL. The non-radioactive LDH cytotoxicity assay (Promega) was used to evaluate the specific lysis of TAPA-presenting DC.

### DETAILED DESCRIPTION

This invention provides methods and compositions for the treatment and/or prevention of cancer, including solid malignancies and hematologic malignancies. In particular, the present invention relates to immunotherapy using antigen-presenting cells loaded with tumor associated peptide antigens (TAPAs). If a subject expresses at least one tumor-associated antigen, the subject can be treated by antigen-presenting cells (e.g., dendritic cells) loaded with at least one tumor-associated peptide antigen (TAPA) derived from at least one tumor-associated antigen expressed by the subject. This personalized immunotherapy induces or enhances immune responses to cells that express at least one tumor-associated antigen.

Encompassed by the present invention is a method of treating and/or preventing cancer in a subject. The method may contain the following steps: (a) determining whether the subject expresses at least one tumor-associated antigen; (b) loading antigen-presenting cells with at least one tumor-associated peptide antigen (TAPA) derived from at least one tumor-associated antigen expressed by the subject, or introducing into antigen-presenting cells nucleic acids encoding at least one tumor-associated antigen expressed by the subject; and (c) administering the antigen-presenting cells from step (b) to the subject.

### Tumor-Associated Antigens

The present methods and systems may target any tumor-associated antigen that is an antigenic substance produced in tumor cells. Non-limiting examples of tumor antigens include cancer-testis (CT) antigens, epidermal growth factor receptor (EGFR, such as Her-2/neu), Ropporin, PTTG1, Span-xb, HM1.24, mucins (e.g., mucin 16 or MUC16, also known as CAl25), human epididymis protein 4 (HE4), Beta human chorionic gonadotropin (beta-hCG), urinary gonadotropin fragment, Alpha-fetoprotein (AFP), Inhibin, estradiol, carcinoembryonic antigen (CEA), squamous cell carcinoma (SCC) antigen, Müllerian inhibiting substance (MIS), topoisomerase II, Carbohydrate antigen 19-9, Cancer antigen 27-29, human telomerase reverse transcriptase (hTERT), ferritin, lysophosphatidic acid, MIB1-determined tumor growth fraction, Ll (CAM), Mesothelin, Osteopontin, Vascular endothelial growth factor (VEGF), Interleukin 8 (IL-8), Macrophage colony-stimulating factor (M-CSF), Insulinlike growth factor-binding protein-3, Tumor-associated trypsin inhibitor, Cyclin E, OVX1, CA-15-3, and CA-19-9.

Cancer-testis (CT) antigens are proteins expressed in normal gametogenic tissues and in different types of tumors (Scanlan et al., 2002, Immunol Rev. 188:22-32. Scanlan et al., 2004, Cancer Immun. 4:1. Zendman et al., 2003, J Cell Physiol. 194(3):272-88. Simpson et al., 2005, Nat Rev Cancer. 5(8):615-25. Bodey, 2002, Expert Opin Biol Ther. 2(6):577-84). In testis, CT antigens are expressed exclusively in cells of the germ cell lineage, although there is a marked variation in the protein expression pattern during different stages of sperm development. Likewise, a heterogeneous expression is also observed in tumors. It has been proposed that the aberrant expression of CT antigens in tumors recapitulates portions of the germline gene expression program and is related to some characteristics of the neoplastic phenotype such as immortality, invasiveness, immune evasion and metastatic capacity. Chiriva-Internati et al., Cancer Testis Antigens: A Novel Target in Lung Cancer, International Reviews of Immunology, 31:321-343, 2012. Pandey et al., Cancer Testes Antigens in Breast Cancer: Biological Role, Regulation, and Therapeutic Applicability, International Reviews of Immunology, 31:302-320, 2012. Mirandola et al., Cancer Testis Antigens: Novel Biomarkers and Targetable Proteins for Ovarian Cancer, International Reviews of Immunology, 30:127-137, 2011. Simpson et al., 2005, Nat Rev Cancer. 5(8):615-25. Old, L J., 2001, Cancer Immun. 1:1. U.S. Pat. No. 8,207,300.

Non-limiting examples of CT antigens include SP17, ASP, NY-ESO (e.g., NY-ESO-1, etc.) CABYR, TSP50, BORIS, RQCD1, BAGE, SSX, SCP-1, Piwil2, OY-TES-1, LAGE-1, AKAP (e.g., AKAP-4 etc.), SCP-1/HOM-TES-14, MAGE (e.g., MAGE-1, MAGE-A, MAGE-B, MAGE-C, etc.), GAGE (GAGE-A, GAGE-B, etc.), PAGE, XAGE, CAGE, HOM-TES-85, SAGE, BAGE, CT9/3BRDT, HAGE, SPO11, and SPAG9.

In one embodiment, the present method comprises the step of determining whether a subject expresses at least one tumor-associated antigen, e.g., selected from Sp17, Ropporin, AKAP-4, PTTG1, Span-xb, Her-2/neu, HM1.24, NY-ESO-1, MAGE-1 and combinations thereof.

Sperm protein 17 (Sp17) is a highly immunogenic spermatozoan protein which has been considered a potential therapeutic target for immunocontraception in the last few years [25]. We have identified Sp17 as a novel cancer-testis antigen (CTA) in hematologic and SM. Using RT-PCR, Sp17 transcripts were detected in more than 70% of tumor-cell enriched bone marrow from multiple myeloma (MM) patients. Sp17 gene expression was associated with Sp17 protein translation as demonstrated by Western blot analysis. Northern blot analysis and RT-PCR, demonstrated Sp17 expression in normal testis and tumor cells, but not in normal tissues, suggesting Sp17 could serve as an immunotherapeutic target in MM [26]. We have generated Sp17-specific HLA-class I restricted cytotoxic T lymphocyte (CTL) after pulsing monocyte-derived DCs with recombinant Sp17 protein [14]. These Sp17-specific CTLs were able to lyse autologous target cells pulsed with recombinant Sp17 protein as well as Sp17-expressing fresh MM cells. Moreover, we have generated a synthetic Sp17 peptide (103-111) (ILDSSEEDK) capable of inducing HLA-A1 restricted CTL recognition and activation [27]. We have also expanded our evaluation of Sp17 potential as an immunotherapeutic target by recently demonstrating its strong immunogenicity in an ovarian cancer animal model [28]. Moreover, we have recently found Sp17 is expressed in human lung cancer cell lines and tumor tissues, but not in normal lung [29]. More importantly, we have shown Sp17-loaded DCs activate CTLs capable of eliciting antitumor responses, in vitro [29]. Dadabayev et at have reported the safety and clinical response of Sp17-pulsed DCs in patients with MM and ovarian cancer, validating the clinical potential of this protein as an immunotherapeutic target [30].

A-kinase anchoring protein 4 (AKAP-4) is a member of a family of scaffolding proteins involved in the control of signal transduction by targeting cyclic adenosine monophosphate-dependent protein kinase-A, and directing its actions [31, 32]. We have shown that AKAP-4 is expressed in lung cancer and MM (multiple myeloma) cells, at both the transcriptional and the protein level, with no evidence of expression in human normal tissues, other than the testis [29, 33]. The lack of expression in normal tissues and the presence of AKAP-4 antibodies in lung cancer and MM patient sera supports the protein's immunogenicity and indicates AKAP-4 is a tumor-associated antigen. Additionally, we have also demonstrated AKAP-4 serves as a marker of disease status in a murine model of MM [34]. Thus, AKAP-4 is a potential target for developing specific immunotherapeutic strategies against MM, lung cancer and other SM [29, 35].

Ropporin is a rhophilin-associated protein normally expressed in the inner fibrous sheath of sperm flagella. Ropporin has previously been found to interact with other fibrous sheath proteins, including Sp17 and AKAP-110, suggesting a common or related biological function. A study by Li et at has demonstrated a very restricted RNA expression of ropporin in normal tissues, with the exception of testicular and fetal liver tissue [36]. Ropporin expression was also detected in tumor cells derived from the bone marrow in 6 of 16 (37.5%) patients with MM, 6 of 14 (43%) cases of CLL and 2 of 11 (18%) cases of acute myeloid leukemia. No ropporin transcripts were detected in the peripheral blood mononuclear cells of 17 healthy donors. Importantly, these investigators detected high titers of antibodies against ropporin in 8 of 30 MM (26.7%), 7 of 24 AML (29.2%), 18 of 31 chronic lymphocytic leukemia (CLL) (58.1%), 9 of 27 chronic myelogenous leukemia (CML) (33.3%) and 1 of 3 acute lymphoblastic leukemia (ALL) (33.3%), compared to healthy donors. The presence of ropporin antibodies in patients with a variety of malignancies indicates this CTA is a highly immunogenic protein. Taken together, these findings support the notion that ropporin is a tumor-restricted CTA with a potential role in the biology of certain neoplasms. The restricted expression of ropporin in neoplastic cells, testes and fetal liver, makes it a suitable candidate as a target for immunotherapy.

PTTG-1 is a novel oncogene involved in transcriptional and cell cycle regulation with expression in the normal testis and thymus [37]. PTTG-1 has been shown to be highly expressed in different hematologic malignancies (HM) including promyelocytic leukemia (PML) cell line HL-60, CML cell line K-562, ALL cell line MOLT-4 and Burkitt's lymphoma cell line Raji [38]. PTTG-1 has also been shown to be associated with tumorigenesis, angiogenesis and cancer progression, making it a logical therapeutic target [37]. We and others have found PTTG-1 is expressed at the transcriptional level in MM, with PTTG-1 being expressed in 63% of MM patients and 66% of human MM cell lines studied, but not in normal tissues [39]. We also documented cytoplasmic and surface PTTG-1 expression in plasma cells from MM patients supporting PTTG-1 potential role as a therapeutic target in patients with HM. More recently, we have also demonstrated PTTG-1 expression in lung cancer tissues and cell lines, and showed PTTG-1-loaded DCs can activate CTL-mediated lysis of human lung cancer cells, in vitro [29]. Therefore, our data indicates the suitability of using PTTG-1 as a potential target for immunotherapy in both HM and SD.

Span-xb is a novel CTA expressed in CML and other HM. Using RT-PCR, we have detected Span-xb transcripts in 20% of MM patients, 33% of patients with CLL, 29% of CML patients and 50% of patients with AML. In contrast, Span-xb expression was not detected in peripheral blood or bone marrow samples from healthy donors [40]. Importantly, span-xb gene expression has also been found in a variety of SM, including melanoma and carcinomas of the lung, colon and breast, making it a target for immunotherapeutic interventions [41].

HER-2/neu is a trans-membrane tyrosine-kinase involved in aberrant signal transduction in a variety of neoplasms [42, 43]. HER-2/neu amplification has been demonstrated in certain HM and its functional inhibition, using anti-sense oligonucleotides, results in a reduced tumor cell proliferative rate. The observation that Her-2/neu is expressed at very low levels in normal tissues and preferentially expressed in neoplastic cells, suggest it could serve as an immunotherapeutic target in HER-2/neu-expressing SM. In fact, Scardino et al have demonstrated HLA-restricted CTL activation by high several high affinity HER-2/neu peptides capable of HLA-restricted killing of neoplastic cells of diverse origin [44]. Thus, specific Her-2/neu peptides may serve as good candidates for immunotherapy in HER-2-expressing SM.

HM1.24 is a novel, 29-33 kDa membrane glycoprotein expressed in mature B-cells. We have proposed HM1.24 as a new antigen for CTLs activation against MM [45]. HM1.24 expression has been found in all five human MM cell lines assayed, as well as in mature, Ig-secreting B-cells (plasma cells and lymphoplasmacytoid cells), but not in non-B-Cells in the peripheral blood, bone marrow, liver, spleen, kidney, or heart of normal individuals or patients with non-plasma-cell-related malignancies. Thus, HM1.24 protein represents a specific marker of late-stage B-cell maturation and may potentially serve as a target antigen for the development of immunotherapeutic strategies specific against MM. HM1.24 is also expressed in SM including brain tumors, renal, hepatocellular, breast, ovarian, and breast carcinomas, with some expression in a few normal organs including liver and kidney [46]. Although HM1.24 function is unknown at this time, its promise as a therapeutic target has been demonstrated using a specific HM1.24 monoclonal antibody (MoAb)[47].

NY-ESO-1 is one of the most immunogenic tumor antigens known to date. Spontaneous humoral and cellular immune responses against NY-ESO-1 are detected in a substantial proportion of patients with NY-ESO-1 expressing malignancies and NY-ESO-1 antibody titers correlate with clinical development of disease [48]. Moreover, the development of NY-ESO-1 serum antibody is associated with detectable NY-ESO-1-specific CD8+ T cell reactivity, suggesting this antigen is an excellent immunogen and potential therapeutic target, in vivo [49].

MAGE-1 is expressed in HM, including human MM cell lines and malignant plasma cells, as well as melanomas [50, 51]. Both RNA and protein expression has been demonstrated in MM cells, but not in polyclonal, reactive plasma cells. Moreover, anti-MAGE-1 HLA-A1 cytotoxic T lymphocytes can efficiently kill MAGE-1 HLA-A1 expressing MM and melanoma cells, suggesting MAGE-1 represents a specific and potential immunotherapeutic target for patients with these malignancies.

A tumor-associated peptide antigen (TAPA) is a peptide derived from a tumor-associated antigen as described herein. For example, a TAPA may be an immunogenic fragment of a tumor-associated antigen. In one embodiment, the TAPA is MHC class I molecule-restricted. The MHC class I molecules may be HLA-A (e.g., HLA-A1, HLA-A2), HLA-B, HLA-C, HLA-E, HLA-F, HLA-G, HLA-K, or HLA-L. Non-limiting examples of TAPAs include SEQ ID NOs: 1, 2, 3, 4, 5, 6, 7, 8, or combinations thereof (Table 1). A TAPA may also be an immunogenic peptide at least 60%, at least 70%, at least 80%, at least 85%, at least 90%, at least 95% identical to a fragment of a tumor-associated antigen (e.g., a fragment of a tumor-associated antigen that may be SEQ ID NOs: 1, 2, 3, 4, 5, 6, 7, 8, or combinations thereof).

TABLE 1

| | | SEQ |
|---|---|---|
| Name | Sequence | ID NO. |
| Sp17(103-111) | ILDSSEEDK | 1 |
| AKAP-4(150-158) | YADQVNIDY | 2 |
| PTTG-1(70-78) | ATEKSVKTK | 3 |
| Ropporin(113-121) | FTEEIEWLK | 4 |
| Span-Xb(86-94) | DQMEEEEFI | 5 |
| Her-2/neu(106-114) | QLFEDNYAL | 6 |
| HM1.24(9-17) | FLVFVFVFL | 7 |
| NYESO-1(159-167) | LMWITQCFL | 8 |
| MAGE-1(248-256) | TLAKFSPYL | 9 |

A TAPA may comprise or consist of 8 to 10 amino acid residues, 9 amino acid residues, 6 to 30 amino acid residues, 7 to 25 amino acid residues, 8 to 20 amino acid residues, 10 to 20 amino acid residues, 11 to 18 amino acid residues, 12 to 16 amino acid residues, or 13 to 15 amino acid residues.

In one embodiment, the TAPA is capable of eliciting an immune response (e.g., a cellular immune response) to a cancer cell, a precancerous cell, or a cell-type predisposed to developing cancer. In some embodiments, the peptides are capable of directing human cytotoxic T lymphocyte (CTL) to recognize and lyse a cancer cell, a precancerous cell, or a cell-type predisposed to developing cancer.

### Assaying Expression Levels of Tumor-Associated Antigens

In certain aspects, the present method contains the step of screening a patient or patient tumor for expression of one or more of the tumor-associated antigens. Either the RNA (e.g., mRNA) or protein level of a tumor-associated antigen may be assayed. The step of determining the level of the tumor-associated antigen or its nucleic acid molecules (mRNA, DNA, etc.) may include contacting the biological sample with an agent that selectively binds to the tumor-associated antigen or the nucleic acid. U.S. Pat. No. 7,670,599. The detected expression level in the test sample may be compared to a reference sample from, e.g., a healthy subject(s).

### Protein-Based Assays

The level of a tumor-associated antigen protein can be detected and/or quantified by any of a number of methods well known to those of skill in the art. The methods may include various immunoassays such as immunohistochemistry, enzyme-linked immunosorbent assay (ELISA), antibody sandwich capture assay, immunofluorescent assay, Western blot, enzyme-linked immunospot assay (EliSpot assay), precipitation reactions (in a fluid or gel), immunodiffusion, immunoelectrophoresis, radioimmunoassay (RIA), and the like. Also included are analytic biochemical methods such as electrophoresis, capillary electrophoresis, high performance liquid chromatography (HPLC), thin layer chromatography (TLC), hyperdiffusion chromatography, and the like. U.S. Pat. Nos. 4,366,241; 4,376,110; 4,517,288; and 4,837,168. Methods in Cell Biology Volume 37: Antibodies in Cell Biology, Asai, ed. Academic Press, Inc. New York (1993); Basic and Clinical Immunology 7th Edition, Stites & Terr, eds. (1991).

The level of a tumor-associated antigen may be detected by using molecules (e.g., polypeptides, etc.) that bind to the tumor-associated antigen. For example, the binding polypeptide may be an antibody or antibody fragment, such as an Fab, F(ab)₂, F(ab′)₂, Fd, or Fv fragment of an antibody. Any of the various types of antibodies can be used for this purpose, including, but not limited to, polyclonal antibodies, monoclonal antibodies, humanized antibodies, human antibodies (e.g., generated using transgenic mice, etc.), single chain antibodies (e.g., single chain Fv (scFv) antibodies), heavy chain antibodies and chimeric antibodies. The antibodies can be from various species, such as rabbits, mice, rats, goats, chickens, guinea pigs, hamsters, horses, sheep, llamas etc.

The antibodies can be prepared by any suitable methods, including administering a protein, fragments of a protein, cells expressing the protein or fragments thereof and the like to an animal to induce polyclonal antibodies. The present invention also provides methods of producing monoclonal antibodies to the tumor-associated antigens described herein. The production of monoclonal antibodies is performed according to techniques known in the art.

In one embodiment, immunohistochemistry is used to assay the tumor-associated antigen level. In one embodiment, antibodies that specifically bind to a tumor-associated antigen are contacted with a tissue sample (e.g., a histological sample). Those antibodies that specifically bind to the sample are visualized, or otherwise detected, and provide an indication of the location, presence, absence or quantity of the tumor-associated antigen in the sample. The antibodies are typically detected by detection of a label either affixed to the antibody or subsequently added after the tissue contacting step.

In another embodiment, Western blot (immunoblot) is used to detect and quantify a tumor-associated antigen in a sample. The technique may comprise separating sample proteins by gel electrophoresis, transferring the separated proteins to a suitable solid support, and incubating the sample with the antibodies that specifically bind the tumor-associated antigen.

The invention further includes protein microarrays (including antibody arrays) for the analysis of expression of CT antigens. Protein microarray technology, which is also known as protein chip technology and solid-phase protein array technology, is well known to those of ordinary skill in the art. Protein microarray may be based on, but not limited to, obtaining an array of identified peptides or proteins on a fixed substrate, binding target molecules or biological constituents to the peptides, and evaluating such binding. See, e.g., MacBeath et al., Printing Proteins as Microarrays for High-Throughput Function Determination, Science 289(5485):1760-1763, 2000. The tissue may be obtained from a subject or may be grown in culture (e.g., from a cell line). In some embodiments of the invention, one or more control peptide or protein molecules are attached to the substrate.

The polypeptides that may be used to assay the level of a tumor-associated antigen may be derived also from sources other than antibody technology. For example, such binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptides and non-peptide synthetic moieties. The tumor-associated antigens can be used to screen peptide libraries, including phage display libraries, to identify and select peptide binding partners of the tumor-associated antigens. Yeast two-hybrid screening methods also may be used to identify polypeptides that bind to the tumor-associated antigens.

### Nucleic Acid-Based Assays

The present methods may also assay the presence of or quantity the tumor-associated antigen gene or gene product. Gene products include nucleic acids (e.g. mRNAs) derived from the gene.

The level of the DNA or RNA (e.g., mRNA) molecules may be determined using routine methods known to those of ordinary skill in the art. The measurement result may be an absolute value or may be relative (e.g., relative to a reference oligonucleotide, relative to a reference mRNA, etc.). The level of the nucleic acid molecule may be determined by nucleic acid hybridization using a nucleic acid probe, or by nucleic acid amplification using one or more nucleic acid primers.

Nucleic acid hybridization can be performed using Southern blots, Northern blots, nucleic acid microarrays, etc.

For example, the DNA encoding a tumor-associated antigen in a sample may be evaluated by a Southern blot. Similarly, a Northern blot may be used to detect a tumor-associated antigen mRNA. In one embodiment, mRNA is isolated from a given cell sample, and then electrophoresed to separate the mRNA species. The mRNA is transferred from the gel to a solid support. Labeled probes are used to identify or quantity CT antigen nucleic acids.

In certain embodiments, labeled nucleic acids are used to detect hybridization. Complementary nucleic acids may be labeled by any one of several methods typically used to detect the presence of hybridized polynucleotides. One method of detection is the use of autoradiography. Other labels include ligands that bind to labeled antibodies, fluorophores, chemiluminescent agents, enzymes, and antibodies which can serve as specific binding pair members for a labeled ligand.

Nucleic acid microarray technology, which is also known as DNA chip technology, gene chip technology, and solid-phase nucleic acid array technology, may be based on, but not limited to, obtaining an array of identified nucleic acid probes on a fixed substrate, labeling target molecules with reporter molecules (e.g., radioactive, chemiluminescent, or fluorescent tags such as fluorescein, Cye3-dUTP, or Cye5-dUTP, etc.), hybridizing target nucleic acids to the probes, and evaluating target-probe hybridization. Jackson et al. (1996) Nature Biotechnology, 14: 1685-1691. Chee et al. (1995) Science, 274: 610-613.

The level of a tumor-associated antigen nucleic acid (e.g., RNA, DNA) may be assayed by in situ hybridization. Angerer et al. (1987) Methods Enzymol., 152: 649-660. In one embodiment, tissues or cells are denatured. The tissues or cells are then contacted with a hybridization solution at a moderate temperature to permit annealing of labeled probes specific to the tumor-associated antigen nucleic acids. The probes may be labeled with molecules as discussed herein.

The sensitivity of the hybridization assays may be enhanced through use of a nucleic acid amplification system that multiplies the target nucleic acid being detected.

Nucleic acid amplification assays include, but are not limited to, the polymerase chain reaction (PCR), reverse transcription polymerase chain reaction (RT-PCR), real-time RT-PCR, quantitative RT-PCR, etc.

Measuring or detecting the amount or level of mRNA in a sample can be performed in any manner known to one skilled in the art and such techniques for measuring or detecting the level of an mRNA are well known and can be readily employed. A variety of methods for detecting mRNAs have been described and may include, Northern blot, microarrays, real-time PCR, RT-PCR, targeted RT-PCR, in situ hybridization, deep-sequencing, single-molecule direct RNA sequencing (RNAseq), bioluminescent methods, bioluminescent protein reassembly, BRET (bioluminescence resonance energy transfer)-based methods, fluorescence correlation spectroscopy and surface-enhanced Raman spectroscopy (Cissell, K. A. and Deo, S. K. (2009) Anal. Bioanal. Chem., 394:1109-1116).

The methods of the present invention may include the step of reverse transcribing RNA when assaying the level or amount of an mRNA.

These assays of determining the presence and/or level of the molecules of the invention in cells and tissues may include use of labels to monitor the presence of the molecules of the invention. The labels can be any material having a detectable physical or chemical property. Thus, a label is any composition detectable by spectroscopic, photochemical, biochemical, immunochemical, electrical, optical or chemical means. Such labels may include, but are not limited to, a fluorescent label, a radiolabel, a chemiluminescent label, an enzyme, a metallic label, a bioluminescent label, a chromophore, biotin etc. For example, a fluorescently labeled or radiolabeled antibody that selectively binds to a polypeptide of the invention may be contacted with a tissue or cell to visualize the polypeptide. In some aspects of the invention, a label may be a combination of the foregoing molecule types.

### Antigen-Presenting Cells (APCs)

The APCs of the present invention may be loaded with at least one tumor-associated peptide antigen (TAPA) derived from at least one tumor-associated antigen expressed by the patient. Alternatively or simultaneously, nucleic acids encoding at least one tumor-associated antigen expressed by the patient may be introduced into the APCs.

Non-limiting examples of antigen-presenting cells including dendritic cells, macrophages, B cells, cells of myeloid lineage, Langerhans cells, epithelial cells, or any nucleated cells.

The APC may be autologous or allogeneic. The APC may be isolated from a subject. The APC may also be derived from cells isolated from a subject.

In some embodiments, the present invention provides a method for eliciting in a subject an immune response to a cell that expresses a tumor-associated antigen, the method comprising administering to the subject antigen-presenting cells (e.g., dendritic cells) loaded with at least one tumor-associated antigen, or administering to the subject antigen-presenting cells comprising nucleic acids encoding at least one tumor-associated antigen, wherein the antigen-presenting cells (e.g., dendritic cells), when administered to the subject, elicits the immune response to the cell.

The tumor-associated antigen-loaded antigen-presenting cells may also be used to activate T lymphocytes. As described herein, the present antigen-presenting cells and/or T lymphocytes may be used for prophylactic or therapeutic applications.

### Dendritic Cells (DCs)

DCs can be generated in vivo or ex vivo from immature precursors (e.g., monocytes). For example, for ex vivo DC generation, a cell population enriched for DC precursor cells (e.g., peripheral blood mononuclear cells (PBMCs)) is obtained from a patient, and then the DC precursor cells are differentiated ex vivo into mature DCs. Typically, to generate immature dendritic cells (DC), one must first purify or enrich the monocytic precursors from other cell types. For example, peripheral blood mononuclear cells (PBMCs) are extracted from whole blood (e.g., over Ficoll density gradient centrifugation). Then the PBMCs will be used to generate monocytic dendritic cell precursors.

DCs can be generated from monocytes, CD34+ cells (i.e., cells expressing CD34), etc.

In certain embodiments, monocytic dendritic cell precursors are isolated by adherence to a monocyte-binding substrate. For example, a population of leukocytes (e.g., isolated by leukapheresis) can be contacted with a monocytic dendritic cell precursor adhering substrate. When the population of leukocytes is contacted with the substrate, the monocytic dendritic cell precursors in the leukocyte population preferentially adhere to the substrate. In one embodiment, monocytes are isolated through adherence of the monocytic precursors to a plastic (polystyrene) surface, as the monocytes have a greater tendency to stick to plastic than other cells found in, for example, peripheral blood, such as lymphocytes and natural killer (NK) cells.

Methods for isolating cell populations enriched for dendritic cell precursors and immature dendritic cells from various sources, including blood and bone marrow, are known in the art. For example, dendritic cell precursors and immature dendritic cells can be isolated by phlebotomy, by apheresis or leukapheresis, by collecting heparinized blood, by preparation of buffy coats, rosetting, centrifugation, density gradient centrifugation (e.g., using Ficoll, Percoll (colloidal silica particles of 15-30 mm diameter coated with polyvinylpyrrolidone (PVP)), sucrose, and the like), differential lysis of cells, filtration, and the like. In one embodiment, dendritic cell precursors can be selected using CD14 selection of G-CSF mobilized peripheral blood. U.S. Pat. No. 8,728,806.

Before the subject's blood or bone marrow is obtained to isolate dendritic cell precursors, the subject may be administered granulocyte macrophage colony stimulating factor (GM-CSF) to increase bone marrow production of monocytes and dendritic cell precursors. In certain embodiments, GM-CSF is administered at a dose ranging from about 10 μg/day to about 500 μg/day, from about 20 μg/day to about 300 μg/day, from about 50 μg/day to about 250 μg/day, from about 100 μg/day to about 300 μg/day, from about 200 μg/day to about 300 μg/day, about 200 μg/day, or about 250 μg/day. The dose of GM-CSF can also be lower or higher. In certain embodiments, GM-CSF may be administered for about 1 day, about 2 days, about 3 days, about 4 day, about 5 days, about 6 days, about 1 week, about 1.5 weeks, about 2 weeks, or longer.

The effect of GM-CSF may be potentiated by another immunostimulant (such as plerixafor).

Variations on this method include different methods of purifying monocytes, including, for example, tangential flow filtration (TFF), or by binding antibodies attached to beads to surface molecules on the monocytes. The beads with the bound cells are then concentrated in a column, or on a magnetic surface, such that contaminating cells can be washed away, after which the monocytes are eluted off the beads. In yet another method to obtain dendritic cells precursors, cells expressing the stem cell marker CD34, either from blood (U.S. Pat. No. 5,994,126, incorporated herein by reference) or from the bone marrow are purified. These cells can be cultured with the essential cytokine GM-CSF to differentiate into immature DC. These DC apparently have very similar characteristics and functional properties as immature DC generated from monocytes.

Isolated dendritic cell precursors can be cultured ex vivo for differentiation, maturation and/or expansion. In certain embodiments, the monocytic dendritic cells precursors are differentiated to form immature dendritic cells. The end result of this process is a cell which expresses T cell costimulatory molecules, as well as high levels of molecules of the major histocompatibility complex (MHC), but does not express the dendritic cell maturation marker CD83. These cells are similar to Langerhans cells in the skin, and their prime physiological function is to capture invading microorganisms.

The dendritic cell precursors and/or immature dendritic cells can be cultured and differentiated in suitable culture conditions. The tissue culture media can be supplemented with, e.g., plasma, serum, amino acids, vitamins, cytokines (e.g., granulocyte-macrophage colony-stimulating factor (GM-CSF), interleukins such as Interleukin 4 (IL-4), Interleukin 13 (IL-13), Interleukin 15 (IL-15), or combinations thereof), purified proteins (such as serum albumin), divalent cations (e.g., calcium and/or magnesium ions), growth factors, and the like, to promote differentiation of the cells. Sallusto et al., J. Exp. Med., 179:1109-1118, 1994, incorporated herein by reference. For example, the use of type I interferons and Toll-like receptor agonists to induce DC maturation ex vivo have been shown to stimulate generation of immunogenic, rather than tolerogenic, DCs [58, 59]. In certain embodiments, the blood plasma or serum can be heat-inactivated. The plasma or serum can be autologous, allogeneic or heterologous to the cells. In certain embodiments, the dendritic cell precursors can be cultured in the serum-free media. Such culture conditions can optionally exclude any animal-derived products. In one embodiment, a dendritic cell culture medium contains about 200 units/ml to about 1500 units/ml (e.g., about 1000 units/ml, about 500 units/ml, etc.) of GM-CSF and about 200 units/ml to about 1500 units/ml (e.g., about 800 units/ml, about 500 units/ml, etc.) IL-4.

### Loading of APCs

Immature DC have a high capacity for taking up and processing antigen, but have a limited ability to initiate immune responses. The ability to initiate an immune response is acquired by maturation of the immature DC. This maturation is also referred to as activating, or activation of, the DC. The maturation process may be initiated and/or induced through contact with maturation-inducing cytokines, tumor-associated antigens or tumor-associated peptide antigens and/or nucleic acids encoding tumor-associated antigens or tumor-associated peptide antigens, and the like, as described herein.

APCs (e.g., dendritic cells) can be loaded with one or more peptide antigens (e.g, a tumor-associated peptide antigen). In certain aspects, 1, 2, 3, 4, 5, 6, 7, 8, 9, or more tumor-associated peptide antigens using any combinations of the tumor-associated peptide antigens of Table 1. As used herein, a cell or membrane bound composition (e.g., a liposome) “loaded” (or “pulsed”) with a peptide shall mean that the cell or membrane bound composition has been incubated with the peptide under conditions permitting entry into and/or attachment onto the cell or membrane bound composition of the peptide. For example, APCs (e.g., dendritic cells) can be incubated with one or more tumor-associated peptide antigens under conditions that are needed to load the MHC of the APC (e.g., the dendritic cell). Suitable conditions for antigen loading are provided that permit an APC to contact, process and/or present one or more antigens on its MHC, whether intracellularly or on the cell surface. The incubation time may range from about 10 minutes to about 3 days or longer, from about 30 minutes to about 36 hours, from about 1 hour to about 28 hours, from about 2 hours to about 24 hours, from about 4 hours to about 24 hours, from about 4 hours to about 16 hours, from about 16 hours to about 24 hours, from about 20 hours to about 28 hours, from about 2 hours to about 4 hours, from about 1 hour to about 12 hours, from about 2 hours to about 8 hours, from about 3 hours to about 5 hours, for less than about a week, illustratively, for about 1 minute to about 48 hours, about 2 minutes to about 36 hours, about 3 minutes to about 24 hours, about 4 minutes to about 12 hours, about 6 minutes to about 8 hours, about 8 minutes to about 6 hours, about 10 minutes to about 5 hours, about 15 minutes to about 4 hours, about 20 minutes to about 3 hours, about 30 minutes to about 2 hours, about 40 minutes to about 1 hour, about 16 hours, about 20 hours, about 24 hours, about 28 hours, about 1 hour, about 2 hours, or about 4 hours. The incubation temperature may range from about 4° C. to about 37° C., from about 25° C. to about 37° C., about 4° C., about 25° C., or about 37° C.

The concentration of the peptide for loading may range from about 1 μg/ml to about 1 mg/ml, from about 5 μg/ml to about 800 μg/ml, from about 10 μg/ml to about 600 μg/ml, from about 15 μg/ml to about 400 μg/ml, from about 10 μg/ml to about 200 μg/ml, from about 10 μg/ml to about 100 μg/ml, from about 50 μg/ml to about 100 μg/ml, from about 20 μg/ml to about 100 μg/ml, about 10 μg/ml, about 20 μg/ml, about 30 μg/ml, about 50 μg/ml, about 60 μg/ml, about 80 μg/ml, or about 100 μg/ml.

In one embodiment, one or more tumor-associated antigen can be coupled to a cytolysin to enhance the transfer of the antigens into the cytosol of an antigen-presenting cell for delivery to the MHC class I pathway. Exemplary cytolysins include saponin compounds such as saponin-containing Immune Stimulating Complexes (ISCOM5), pore-forming toxins (e.g., an alpha-toxin), and natural cytolysins of gram-positive bacteria such as listeriolysin O (LLO), streptolysin O (SLO), and perfringolysin O (PFO).

A number of methods for delivery of antigens to the endogenous processing pathway of antigen-presenting cells may be optionally used. Such methods include, but are not limited to, methods involving pH-sensitive liposomes, coupling of antigens to potent adjuvants, apoptotic cell delivery, pulsing cells onto dendritic cells, delivering recombinant chimeric virus-like particles (VLPs) comprising antigen to the MHC class I processing pathway of a dendritic cell line.

APCs may be contacted with nucleic acids encoding one or tumor-associated antigens under a condition sufficient for the at least one tumor-associated peptide antigen to be presented by the APC. For example, antigen-presenting cells (e.g., dendritic cells) can be transfected with expression vectors or infected with viral vectors for introducing nucleic acids encoding tumor-associated antigens into the APCs.

Expression can be optionally effected by targeting the expression construct to specific cells, such as with a viral vector or a receptor ligand, or by using a tissue-specific promoter, or combinations thereof. Non-limiting viral vectors include adeno-associated viruses, lentiviruses, retroviruses, herpes viruses, adenoviruses, vaccinia viruses, baculoviruses, Fowl pox, AV-pox, modified vaccinia Ankara (MVA) and other recombinant viruses.

The time and amount of antigens, or nucleic acids encoding the antigens, necessary for the antigen presenting cells to process and present the antigens can be determined, for example, by assaying T cell cytotoxic activity in vitro or using antigen-presenting cells as targets of CTLs. Other methods that can detect the presence of antigen on the surface of antigen-presenting cells are also contemplated by the presented invention. The antigen-presenting cells loaded with the antigen can be used to stimulate CTL proliferation in vivo or ex vivo. The ability of the loaded dendritic cells to stimulate a CTL response can be measured by assaying the ability of the effector cells to lyse target cells. For example, the non-radioactive LDH cytotoxicity assay or the europium release assay can be used. Volgmann et al., J. Immunol. Methods 119:45-51, 1989.

Ex vivo or in vitro maturation of DCs can be induced by various maturation factors, including, but not limited to, IL-1β, tumor necrosis factor alpha (TNF-α), interferon alpha (IFN-α), poly (I:C), interferon gamma (IFN-γ), Interleukin 1 beta (IL-β), Interleukin 6 (IL-6), prostaglandin E2 (PGE2), poly-dIdC, vasointestinal peptide (VIP), bacterial lipopolysaccharide (LPS), mycobacteria or components of mycobacteria (such as cell wall constituents), or combinations thereof. Additional maturation factors include, for example, an imidazoquinoline compound, e.g., R848 (WO 00/47719, incorporated herein by reference in its entirety), a synthetic double stranded polyribonucleotide, agonists of a Toll-like receptor (TLR), such as TLR3, TLR4, TLR7 and/or TLR9, a sequence of nucleic acids containing unmethylated CpG motifs known to induce the maturation of DC, and the like. Further, a combination of any of the above agents can be used in inducing the maturation of immature dendritic cells or dendritic precursor cells. In certain embodiments, a dendritic maturation cocktail includes (comprises, consists essentially of, or consists of) IL-1β, TNF-α, IFN-α and poly (I:C).

The maturation factors can be added to the dendritic cells before, during or after peptide loading of the dendritic cells.

Immature dendritic cells are matured to form mature dendritic cells. Mature DCs lose the ability to take up antigen and display up-regulated expression of costimulatory cell surface molecules and various cytokines Maturation of dendritic cells can be monitored by methods known in the art. Mature dendritic cells can be selected by expression of one or more markers. The markers include, but are not limited to, CD86, CD80, CD83, CD58, CD1a, HLA-DR, CD40, CD11c, IL-2-beta, TLR-4 and combinations thereof. The dendritic cells can also be identified as lacking or expressing low levels of markers such as CD14. In one embodiment, mature dendritic cells are identified as being CD80+, CD83+, CD86+, and CD14−. Greater MHC expression leads to an increase in antigen density on the DC surface, while up-regulation of costimulatory molecules CD80 and CD86 strengthens the T cell activation signal through the counterparts of the costimulatory molecules, such as CD28 on the T cells.

Cell surface markers can be detected in suitable assays, such as flow cytometry, immunohistochemistry, and the like. The cells can also be monitored for cytokine production (e.g., by ELISA, FACS, or other immune assay). Dendritic cell precursors, immature dendritic cells, and mature dendritic cells, either primed or unprimed with antigens, can be cryopreserved for use at a later date.

The mature DCs of the invention either can be used immediately after their generation (and, optionally, purification) or stored frozen for future use. In certain embodiments, enough mature DCs or T cells are generated to provide an initial dose for the subject as well as cells that can be frozen and stored for future use if necessary.

In certain embodiments of the adoptive immunotherapy methods described above, the cells of interest (i.e., mature DCs) can be purified prior to administration to the subject. Purification of the cells can be done using a variety of methods known in the art, including methods in which antibodies to specific cell surface molecules are employed. These methods include both positive and negative selection methods. For example, cells generated in vitro can be isolated by staining the cells with fluorescently labeled antibodies to cell surface markers followed by sorting of the cells that express both of these markers on their cell surface using fluorescence activated cell sorting (FACS). These and other purification/isolation methods are well known to those of skill in the art.

In certain other embodiments, mature DCs or T cells can be expanded in vitro from freshly isolated or frozen cell stocks to generate sufficient numbers of cells for effective adoptive immunotherapy. The expansion of the cells can be achieved by any means that maintains their functional characteristics. The phenotypic and functional properties of the resultant expanded cells can be tested prior to their therapeutic use and/or storage to verify that the expansion process has altered their activity.

### Immunotherapy

Methods are provided for administration of mature dendritic cells to a subject in need of immunostimulation. In certain embodiments, such methods are performed by obtaining dendritic cell precursors or immature dendritic cells, differentiating and maturing those cells in the presence of a tumor-associated antigen or a tumor-associated peptide antigen (or a nucleic acid composition) to form a mature dendritic cell population. The immature dendritic cells can be contacted with antigen prior to or during maturation.

The DC administration (vaccination) may be given once, twice, three times, four times, five times, six times, seven times, eight times, nine times, ten times, eleven times, twelve times, thirteen times, fourteen times, fifteen times, or more, within a treatment regime to a subject/patient. The DC administration (vaccination) may be given every 2 days, every 3 days, every 4 days, every 5 days, every 6 days, every 7 days, every 8 days, every 9 days, every 10 days, every 11 days, every 12 days, every 13 days, every 14 days, every 16 days, every 18 days, every 20 days, every 1 month, every 2 months, every 3 months, every 6 months, or at different frequencies.

The DC may be administered at a dose ranging from about 1×10³ DCs to about 1×10¹² DCs, from about 1×10⁴ DCs to about 1×10¹⁰ DCs, from about 1×10⁵ DCs to about 1×10⁹ DCs, from about 1×10⁶ DCs to about 1×10⁸ DCs, from about 1×10⁶ DCs to about 1×10⁷ DCs, from about 1×10⁷ DCs to about 1×10⁸ DCs, about 1×10⁵ DCs, about 1×10⁶ DCs, about 1×10⁷ DCs, about 1×10⁸ DCs, or about 1×10⁹ DCs.

In a related embodiment, the mature dendritic cells can be contacted with, and thus, activate, lymphocytes. The activated, polarized lymphocytes, optionally followed by clonal expansion in cell culture, can be administered to a subject in need of immunostimulation.

The present invention provides a method for eliciting in a subject an immune response to a cell expressing at least one tumor-associated antigen. The method comprises administering to the subject antigen-presenting cells (e.g., dendritic cells) loaded with at least one tumor-associated peptide antigen (or tumor-associated antigen), or antigen-presenting cells (e.g., dendritic cells) comprising nucleic acids encoding at least one tumor-associated antigen, where the antigen-presenting cells, when administered to the subject, elicits an immune response to the cell that expresses at least one tumor-associated antigen.

In some aspects, the present invention provides a method of treating a tumor cell, the method comprising administering to a subject a therapeutically or prophylactically effective amount of a pharmaceutical composition to reduce or inhibit growth or spread of the cell in the subject, wherein the composition comprises: an antigen-presenting cell presenting the at least one tumor-associated peptide antigen, a lymphocyte primed against the tumor-associated antigen, or a combination thereof.

The antigen-presenting cells loaded with one or more tumor-associated peptide antigens may be used to contact lymphocytes under conditions sufficient to produce tumor-associated antigen-specific lymphocyte capable of eliciting an immune response against a tumor cell. Thus, the antigen-presenting cells also can be used to provide lymphocytes, including T lymphocytes and B lymphocytes, for eliciting an immune response against a cell that expresses a tumor-associated antigen. In one embodiment, a preparation of T lymphocytes is contacted with the antigen-presenting cells described above for a period of time, preferably for at least about 24 hours, for priming the T lymphocytes to the at least one tumor-associated antigen presented by the antigen-presenting cells. In another embodiment, a population of antigen-presenting cells can be co-cultured with a heterogeneous population of peripheral blood T lymphocytes together with at least one tumor-associated antigen, or nucleic acids comprising the at least one tumor-associated antigen. The cells can be co-cultured for a period of time and under conditions sufficient for the tumor-associated antigens or their processed forms to be presented by the antigen-presenting cells and the antigen-presenting cells to prime a population of T lymphocytes to respond to cells that express a tumor-associated antigen. Accordingly, T lymphocytes and B lymphocytes that are primed to respond to cells that express a tumor-associated antigen can be prepared.

The ability to induce lymphocytes to exhibit an immune response can be determined by any method including, but not limited to, determining T lymphocyte cytolytic activity in vitro using for example tumor-associated antigen-specific antigen-presenting cells as targets of tumor-associated antigen-specific cytolytic T lymphocytes (CTL); assaying tumor-associated antigen-specific T lymphocyte proliferation; and determining B cell response to cells expressing a tumor-associated antigen using, for example, ELISA methods.

T lymphocytes can be obtained from any suitable source such as peripheral blood, spleen, and lymph nodes. The T lymphocytes can be used as crude preparations or as partially purified or substantially purified preparations, which can be obtained by standard techniques including, but not limited to, methods involving immunomagnetic or flow cytometry techniques using antibodies.

In another embodiment, T cells can be removed from an individual and treated in vitro with the peptide(s), wherein the resulting CTL are reinfused autologously or allogeneically to the subject. In various other embodiments, the peptide(s) of the present invention also may be administered to the subject, or in vitro to T cells, in the form of a nucleic acid vaccine, wherein one or more suitable gene transfer vectors, such as a plasmid or an engineered viral vector that contains DNA encoding the peptide fragment(s), is administered to the subject or to T cells in vitro.

In one aspect, the present invention provides a method of treating a tumor cell, the method comprising administering to a subject antigen-presenting cells, T lymphocytes, or both, where the antigen-presenting cells have been loaded with at least one tumor-associated peptide antigen, or where the antigen-presenting cells comprise nucleic acids encoding at least one tumor-associated antigen, under a condition sufficient for at least one tumor-associated peptide antigen to be presented by the antigen-presenting cells. The T lymphocytes have been contacted with antigen-presenting cells presenting at least one tumor-associated peptide antigen.

Accordingly, the antigen-primed antigen-presenting cells of the present invention and the antigen-specific T lymphocytes generated with these antigen-presenting cells can be used as immunomodulating compositions for prophylactic or therapeutic applications for cancer. In some embodiments, the tumor-associated antigen-primed antigen-presenting cells of the invention can be used for generating CD8+ CTL, CD4+ CTL, and/or B lymphocytes for adoptive transfer to the subject. Thus, for example, tumor-associated antigen-specific CTLs can be adoptively transferred for therapeutic purposes in subjects afflicted with cancer.

The present compositions or methods may function to provide or enhance an immune response. Generally, the immune response can include humoral immune response, cell-mediated immune response, or both. For example, antigen presentation through an immunological pathway involving MHC class II molecules or direct B-cell stimulation can produce a humoral response; and, antigens presented through a pathway involving MHC I molecules can elicit cell-mediated immune response. A humoral response can be determined by a standard immunoassay for antibody levels in a serum sample from the subject receiving the pharmaceutically acceptable composition. A cellular immune response is a response that involves T cells and can be determined in vitro or in vivo. For example, a general cellular immune response can be determined as the T cell proliferative activity in cells (e.g., peripheral blood leukocytes (PBLs)) sampled from the subject at a suitable time following the administering of a pharmaceutically acceptable composition. Following incubation of e.g., PBMCs with a stimulator for an appropriate period, [³H]thymidine incorporation can be determined. The subset of T cells that is proliferating can be determined using flow cytometry. T cell cytotoxicity can also be determined.

The immune response that is elicited or enhanced may be sufficient for prophylactic or therapeutic treatment of a neoplastic disease, or a symptom associated therewith, particularly cancer. Accordingly, a beneficial effect of the present compositions and/or methods will generally at least in part be immune-mediated, although an immune response need not be positively demonstrated in order for the compositions and methods described herein to fall within the scope of the present invention.

The immunological efficacy of the present methods and compositions may be determined based on the Distribution Free Resampling (DFR) method proposed and described by Moodie et al [66].

The release of cytokines (e.g., IFN-γ, TNF-α, and/or IL-17) may be assayed by, e.g., ELISpot assay, to determine immune responses.

In one embodiment, upon challenge, the CD8+ cytotoxic T lymphocytes (CTL) recognize specific tumor associated peptide antigens (TAPA) in conjunction with MHC class I molecules, leading to secretion of interferon-gamma (IFN-γ) or other cytokines and lysis of cells expressing the specific TAPA. In addition, the CD4+ T helper lymphocytes recognize antigenic peptides in conjunction with MHC class II molecules, also leading to the secretion of IFN-γ which in turn affects other aspects of the immune response.

The cytokine ELISPOT (Enzyme-Linked ImmunoSPOT) assay is designed to enumerate cytokine-secreting cells. The assay has the advantage of detecting only activated/memory T cells and has the ability to detect cytokine release in response to antigen by a single cell thereby permitting direct calculation of responder T cell frequencies. The high sensitivity and easy performance, allowing the determination of peptide-reactive T cells without prior in vitro expansion, makes the ELISPOT assay well suited to monitor T cell responses. Tanguay et al., 1994. Lymphokine Cytokine Res. 13: 259. Carter et al., 1997. Curr. Opin. Immunol. 9: 177.

Briefly, cells are incubated in the wells of the ELISPOT plate pre-coated with a high-affinity monoclonal antibody to which the cytokine, produced during incubation, will bind. Subsequently, cells are washed away. Areas in which the cytokines have been bound are detected with a combination of biotinylated anti-cytokine detection antibodies and φ-labeled goat anti-biotin antibodies. The last step in the assay is the addition of a reagent allowing the precipitation of silver on cp revealing the site of cytokine secretion (i.e., spot formation).

Treating a subject using the present compositions and methods may refer to reducing the symptoms of the disease, reducing the occurrence of the disease, reducing the severity of the disease, and/or preventing a disease from occurring. As such, to treat a subject means both preventing disease occurrence (prophylactic treatment) and treating a subject that has a disease (therapeutic treatment). In particular, treating a subject is accomplished by providing or enhancing an immune response in the subject.

One or more antigens or antigenic peptides may be presented by the present antigen-presenting cells (e.g., dendritic cells), including 2, 3, 4, 5, 6, 7, 8, 9, 10 or more antigens or antigenic peptides. Additionally, multiple independently generated DCs can be administered to a subject. Furthermore, administration of DCs to a subject can be done as often as is required to ameliorate the symptoms associated with the disease state.

The antigen-presenting cells and/or lymphocytes described above can be administered to a subject for eliciting or enhancing an immune response, particularly against tumor cells that express at least one tumor-associated antigen. Such cell-based compositions are useful for treating or preventing cancer.

The APCs (e.g., dendritic cells) and/or T lymphocytes may be autologous, allogenic (e.g., from a different donor subject that is MHC matched or mismatched with the recipient subject) or heterologous to the recipient subject. For example, immature dendritic cells can be harvested from an organ donor and treated in vitro with at least one tumor-associated peptide antigen. The resultant allogeneic mature DCs can then be administered to the subject to promote the cure or treatment of disease in that subject. The antigen-presenting cells and/or lymphocytes described above can be administered to a subject, either by themselves or in combination, for eliciting an immune response, particularly for eliciting an immune response to cells that express a tumor-associated antigen. Such cell-based compositions are useful, therefore, for treating or preventing cancer. The cells can be introduced into a subject by any mode that elicits the desired immune response to cells that express a tumor-associated antigen. Furthermore, the antigen-presenting cells and/or lymphocytes can be derived from the subject (i.e., autologous cells) or from a different subject that is MHC matched or mismatched with the subject (e.g., allogeneic).

In certain embodiments, the present methods induces an immune response to a tumor in a patient. Such methods can comprise one or more steps of (a) obtaining monocytes (which may act as monocytic dendritic cell precursors) from a patient; (b) culturing the monocytes (e.g., with specific cytokines) to induce differentiation into immature dendritic cells; (c) differentiating the immature dendritic cells into mature dendritic cells by contacting the immature dendritic cells with at least one tumor-associated peptide antigen (or tumor-associated antigen); and (d) administering the mature dendritic cells to the patient.

### Immunosuppressive Agents

Prior to the present immunotherapy, one or more immunosuppressive agents may be administered to the patient. In one embodiment, the immunosuppressive agent inhibits or decreases the activity of suppressive T-cell populations, such as suppressor regulatory T-cells (Treg).

Immunosuppressive agents are substances that inhibit or prevent activity of the immune system. This includes substances that suppress cytokine production, down-regulate or suppress self-antigen expression, or mask the MHC antigens. Immunosuppressive agents can be glucocorticoids, cytostatics, antibodies, drugs acting on immunophilins, etc. These may be used alone or in combination.

Cytostatics include, but are not limited to, alkylating agents, antimetabolites, etc. Non-limiting examples of alkylating agents include nitrogen mustards (e.g., cyclophosphamide), nitrosoureas, platinum compounds, and others. Non-limiting examples of antimetabolites include folic acid analogues (e.g., methotrexate), purine analogues (e.g., azathioprine and mercaptopurine), pyrimidine analogues (e.g., fluorouracil), protein synthesis inhibitors, cytotoxic antibiotics (e.g., dactinomycin, anthracyclines, mitomycin C, bleomycin, mithramycin, etc.).

Antibodies include polyclonal antibodies and monoclonal antibodies. For example, the antibodies may be T-cell receptor directed antibodies (e.g., CD3-directed antibodies), or IL-2 receptor directed antibodies (e.g., CD25− directed antibodies).

Drugs acting on immunophilins include, but are not limited to, ciclosporin, tacrolimus, sirolimus, etc.

Other immunosuppressive drugs include, interferons (e.g., IFN-β, IFN-γ, etc.), opioids, TNF binding proteins, mycophenolate, and small biological agents (e.g., fingolimod, myriocin etc.).

Non-limiting examples of immunosuppressive agents include 2-amino-6-aryl-5-substituted pyrimidines; mycophenolate mofetil; azathioprine; 6-mercaptopurine; bromocryptine; danazol; dapsone; glutaraldehyde; anti-idiotypic antibodies for MHC antigens and MHC fragments; cyclosporin A; steroids such as corticosteroids and glucocorticosteroids, e.g., prednisone, prednisolone (e.g., prednisolone sodium phosphate), methylprednisolone, and dexamethasone; methotrexate; hydroxycloroquine; chloroquine; sulfasalazine; leflunomide; cytokine or cytokine receptor antagonists including anti-interferon-gamma, -beta, or -alpha antibodies, anti-tumor necrosis factor-alpha antibodies, anti-TNF-alpha immunoadhesin, anti-tumor necrosis factor-beta antibodies, anti-interleukin-2 antibodies and anti-IL-2 receptor antibodies; anti-LFA-1 antibodies, including anti-CD11 and anti-CD18 antibodies; anti-L3T4 antibodies; heterologous anti-lymphocyte globulin; polyclonal or pan-T antibodies, or monoclonal anti-CD3 or anti-CD4/CD4a antibodies; soluble peptide containing a LFA-3 binding domain; streptokinase; TGF-beta; streptodornase; FK506; RS-61443; deoxyspergualin; rapamycin; T-cell receptor (Cohen et al., U.S. Pat. No. 5,114,721); T-cell receptor fragments (Offner et al. Science 251: 430-432 (1991); WO 90/11294; Ianeway, Nature 341:482 (1989); and WO 91/01133); T cell receptor antibodies; cyclophosphamide; dapsone; penicillamine; plasma exchange; or intravenous immunoglobulin (IVIG).

In one embodiment, prior to the present immunotherapy, cyclophosphamide (CYP) may be administered to the patient. Cyclophosphamide (CYP) may exert cytotoxic and/or immunosuppressive effects, depending on the dose used. For example, studies have shown that low-dose cyclophosphamide (CYP) decreases Treg number and functionality [53]. Greten and colleagues evaluated single-agent CYP doses of 150, 250, and 350 mg/m² in patients with hepatocellular carcinoma and reported that the two (2) lower doses induced a decrease in the absolute and relative frequency of Tregs in the blood of patients, and the 250 mg/m² dose impaired suppressor function and showed decreased Treg frequency up to day 71. Alpha-fetoprotein-specific T-cell responses were also induced in the lower treatment arms [54]. In a different study, breast cancer patients received 50 mg CYP orally daily for 3 months. Tregs were reduced within 14 days of treatment and remained decreased until day 42, returning to pretreatment levels by day 84. Interestingly, endogenous breast tumor-reactive T cells were detected in 27% of patients before CYP treatment and increased to 73% on day 14, 80% on day 42, and 88% on day 84, indicating enhanced T-cell function after the use of metronomic doses of CYP [55]. More recently, the use of metronomic CYP combined with active immunotherapy has been reported [56]. In this study, patients with advanced solid tumors were treated with 3 different regimens of low-dose CYP in combination with an oncolytic adenovirus. CYP was given either as oral metronomic (50 mg/day), a single i.v. injection (1,000 mg), or both. Metronomic CYP was given starting 1 week before the adenovirus, and i.v. cyclophosphamide was given 1 hour prior to the adenovirus. All CYP regimens resulted in higher rates of disease control when compared with the rates for the adenovirus vaccine only, and the metronomic groups were most effective in decreasing Treg numbers. Studies are being conducted combining metronomic doses of CYP with active vaccination strategies for a variety of cancers [57].

In certain embodiments, prior to 1, 2, 3, 4, 5, 6, 7, 8, 9 or all of administration of the tumor-associated antigen loaded DCs, the subject will be administered cyclophosphamide at a dose ranging from about 10 mg/day to about 500 mg/day, from about 20 mg/day to about 400 mg/day, from about 30 mg/day to about 300 mg/day, from about 40 mg/day to about 200 mg/day, from about 50 mg/day to about 150 mg/day, from about 40 mg/day to about 120 mg/day, about 150 mg/day, about 50 mg/day, or about 100 mg/day. The dose of cyclophosphamide can also be lower or higher. In certain embodiments, cyclophosphamide may be administered for about 1 day, about 2 days, about 3 days, about 4 day, about 5 days, about 6 days, about 1 week, about 1.5 weeks, about 2 weeks, or longer. In certain embodiments, administration of cyclophosphamide may be started about 1 day, about 2 days, about 3 days, about 4 day, about 5 days, about 6 days, about 1 week, about 1.5 weeks, about 2 weeks, or earlier, prior to administration of the present loaded DCs to the subject. Administration of the present loaded DCs may be within about 1 day, within about 2 days, within about 3 days, within about 4 days, within about 5 days, within about 6 days, after the cyclophosphamide administration.

### Adjuvants

After each vaccination, adjuvants may be administered. The adjuvant may enhance DC migration and activation in vivo. The adjuvant may provide for increased immunogenicity.

Adjuvants include, but are not limited to, immunomodulatory molecules (e.g., cytokines), oil and water emulsions, aluminum hydroxide, glucan, dextran sulfate, iron oxide, sodium alginate, Bacto-Adjuvant, synthetic polymers such as poly amino acids and co-polymers of amino acids, saponin, paraffin oil, and muramyl dipeptide. U.S. Pat. No. 9,011,835.

In one embodiment, the adjuvant is an immunomodulatory molecule. For example, the immunomodulatory molecule can be a cytokine, chemokine, or immunostimulatory agent, or nucleic acids encoding cytokines, chemokines, or immunostimulatory agents designed to enhance the immunologic response.

Cytokines include, but are not limited to, chemokines, interferons, interleukins, lymphokines, tumor necrosis factor, etc. Cytokines such as granulocyte-macrophage colony-stimulating factor (GM-CSF) are known to induce DC development and serve as an immune adjuvant, both in vitro and in vivo [60, 61]. In one embodiment, low-dose GM-CSF is administered to the subject post vaccination to enhance vaccine-based immune stimulation in patients [62-65]. Examples of immunomodulatory cytokines include interferons (e.g., IFN-α, IFN-β and IFN-γ), interleukins (e.g., IL-1, IL-2, IL-3, IL-4, IL-5, IL-6, IL-7, IL-8, IL-9, IL-10, IL-12, IL-15, IL-20, and IL-21), tumor necrosis factors (e.g., TNF-α and TNF-β), erythropoietin (EPO), FLT-3 ligand, gIp10, TCA-3, MCP-1, MIF, MIP-1α, MIP-1β, Rantes, macrophage colony stimulating factor (M-CSF), granulocyte colony stimulating factor (G-CSF), and granulocyte-macrophage colony stimulating factor (GM-CSF), as well as functional fragments of any of the foregoing.

In certain embodiments, GM-CSF is administered at a dose ranging from about 10 μg/day to about 500 μg/day, from about 20 μg/day to about 300 μg/day, from about 50 μg/day to about 250 μg/day, from about 25 μg/day to about 100 μg/day, from about 30 μg/day to about 80 μg/day, from about 100 μg/day to about 300 μg/day, from about 200 μg/day to about 300 μg/day, about 200 μg/day, about 250 μg/day, about 40 μg/day, about 50 μg/day, about 60 μg/day, or about 70 μg/day. The dose of GM-CSF can also be lower or higher. In certain embodiments, GM-CSF may be administered after the administration of the present loaded DCs for about 1 day, about 2 days, about 3 days, about 4 day, about 5 days, about 6 days, about 1 week, about 1.5 weeks, about 2 weeks, or longer. Any immunomodulatory chemokine that binds to a chemokine receptor, i.e., a CXC, CC, C, or CX3C chemokine receptor, also can be used in the context of the present invention. Examples of chemokines include, but are not limited to, Mip1α, Mip-1β, Mip-3α (Larc), Mip-3β, Rantes, Hcc-1, Mpif-1, Mpif-2, Mcp-1, Mcp-2, Mcp-3, Mcp-4, Mcp-5, Eotaxin, Tarc, Elc, I309, IL-8, Gcp-2 Gro-α, Gro-β, Gro-γ, Nap-2, Ena-78, Gcp-2, Ip-10, Mig, I-Tac, Sdf-1, and Bca-1 (Blc), as well as functional fragments of any of the foregoing.

The adjuvant may be expressed from a vector, or may be administered simultaneously or sequentially, in any order.

### Pharmaceutical Compositions

The present invention provides a pharmaceutical composition comprising antigen-presenting cells (or lymphocytes) loaded with at least one tumor-associated peptide antigen, or antigen-presenting cells (or lymphocytes) comprising nucleic acid encoding at least one tumor-associated antigen (or tumor-associated peptide antigen), described herein. In some embodiments, the composition further comprises an adjuvant as described above. When administered to a subject, the pharmaceutical composition elicits or enhance an immune response to a cell expressing the tumor-associated antigen.

In one embodiment, the present pharmaceutical composition comprises antigen-presenting cells contacted in vitro or ex vivo with at least one tumor-associated antigen (or tumor-associated peptide antigen) under a condition sufficient for the at least one tumor-associated peptide antigen to be presented by the antigen-presenting cells. In another embodiment, the present invention provides a composition comprising antigen-presenting cells contacted in vitro with nucleic acids encoding at least one tumor-associated antigen, under a condition sufficient for the at least one tumor-associated peptide antigen to be presented by the antigen-presenting cells.

The pharmaceutically acceptable compositions of the present invention can be useful as vaccine compositions for prophylactic or therapeutic treatment of a neoplastic disease or symptoms thereof, particularly for preventing or treating cancer in the subject.

In certain embodiments, the present invention concerns formulation of one or more dendritic cell compositions disclosed herein in pharmaceutically acceptable carriers for administration to a cell or a subject, either alone, or in combination with one or more other modalities of therapy.

The pharmaceutical composition may further comprise a pharmaceutically acceptable carrier, diluent, or excipient. Techniques for formulating and administering also can be found in Remington's Pharmaceutical Sciences, Mack Publishing Co., Easton, Pa., latest edition.

Pharmaceutically acceptable carriers known in the art include, but are not limited to, sterile water, saline, glucose, dextrose, or buffered solutions. Agents such as diluents, stabilizers (e.g., sugars and amino acids), preservatives, wetting agents, emulsifying agents, pH buffering agents, additives that enhance viscosity, and the like. Preferably, the medium or carrier will produce minimal or no adverse effects.

The pharmaceutical composition may further comprise an adjuvant. Preferably, the adjuvant employed provides for increased immunogenicity. The adjuvant can be one that provides for slow release of antigen (e.g., a liposome), or it can be an adjuvant that is immunogenic in its own right thereby functioning synergistically with antigens. For example, the adjuvant can be a known adjuvant or other substance that promotes nucleic acid uptake, recruits immune system cells to the site of administration, or facilitates the immune activation of responding lymphoid cells. Adjuvants include, but are not limited to, immunomodulatory molecules (e.g., cytokines), oil and water emulsions, aluminum hydroxide, glucan, dextran sulfate, iron oxide, sodium alginate, Bacto-Adjuvant, synthetic polymers such as poly amino acids and co-polymers of amino acids, saponin, paraffin oil, and muramyl dipeptide. U.S. Pat. No. 9,011,835.

In one embodiment, the adjuvant is an immunomodulatory molecule. For example, the immunomodulatory molecule can be a recombinant protein cytokine, chemokine, or immunostimulatory agent or nucleic acid encoding cytokines, chemokines, or immunostimulatory agents designed to enhance the immunologic response.

Examples of immunomodulatory cytokines include interferons (e.g., IFN-α, IFN-β and IFN-γ), interleukins (e.g., IL-1, IL-2, IL-3, IL-4, IL-5, IL-6, IL-7, IL-8, IL-9, IL-10, IL-12, IL-15, IL-20, and IL-21), tumor necrosis factors (e.g., TNF-α and TNF-β), erythropoietin (EPO), FLT-3 ligand, gIp10, TCA-3, MCP-1, MIF, MIP-1α, MIP-1β, Rantes, macrophage colony stimulating factor (M-CSF), granulocyte colony stimulating factor (G-CSF), and granulocyte-macrophage colony stimulating factor (GM-CSF), as well as functional fragments of any of the foregoing.

Any immunomodulatory chemokine that binds to a chemokine receptor, i.e., a CXC, CC, C, or CX3C chemokine receptor, also can be used in the context of the present invention. Examples of chemokines include, but are not limited to, Mip1α, Mip-1β, Mip-3α (Larc), Mip-3β, Rantes, Hcc-1, Mpif-1, Mpif-2, Mcp-1, Mcp-2, Mcp-3, Mcp-4, Mcp-5, Eotaxin, Tarc, Elc, I309, IL-8, Gcp-2 Gro-α, Gro-β, Gro-γ, Nap-2, Ena-78, Gcp-2, Ip-10, Mig, I-Tac, Sdf-1, and Bca-1 (Blc), as well as functional fragments of any of the foregoing.

In some embodiments, the adjuvant is comprised of incomplete Freund's adjuvant (Montanide ISA 51) or Corynebacterium granulosum P40.

The pharmaceutical composition can be administered in a therapeutically or a prophylactically effective amount. Administering the pharmaceutically acceptable composition of the present invention to the subject can be carried out using known procedures, and at dosages and for periods of time sufficient to achieve a desired effect. For example, a therapeutically or prophylactically effective amount of the pharmaceutical composition can vary according to factors such as the age, sex, and weight of the subject. Dosage regime can be adjusted by one of ordinary skill in the art to elicit the desired immune response including immune responses that provide therapeutic or prophylactic effects.

The pharmaceutically acceptable composition can be administered to the subject at any suitable site, for example, a site that is distal to or proximal to a primary tumor. The route of administering can be parenteral, intramuscular, subcutaneous, intradermal, intraperitoneal, intranasal, intravenous (including via an indwelling catheter), via an afferent lymph vessel, or by any other route suitable in view of the neoplastic disease being treated and the subject's condition. Preferably, the dose will be administered in an amount and for a period of time effective in bringing about a desired response, be it eliciting the immune response or the prophylactic or therapeutic treatment of the neoplastic disease and/or symptoms associated therewith.

Administering can be properly timed, and can depend on the clinical condition of the subject, the objectives of administering, and/or other therapies also being contemplated or administered. In some embodiments, an initial dose can be administered, and the subject monitored for an immunological and/or clinical response. Suitable means of immunological monitoring include using patient's peripheral blood lymphocyte (PBL) as responders and neoplastic cells as stimulators. An immunological reaction also can be determined by a delayed inflammatory response at the site of administering.

One or more doses subsequent to the initial dose can be given as appropriate, typically on a monthly, semimonthly, or a weekly basis, until the desired effect is achieved. Thereafter, additional booster or maintenance doses can be given as required, particularly when the immunological or clinical benefit appears to subside.

Single or multiple administrations of the antigen-presenting cells and lymphocytes can be carried out with cell numbers and treatment being selected by a care provider (e.g., a physician). Preferably, the antigen-presenting cells and/or lymphocytes are administered in a pharmaceutically acceptable carrier. Suitable carriers can be the growth medium in which the cells were grown, or any suitable buffering medium such as phosphate buffered saline. The cells can be administered alone or as an adjunct therapy in conjunction with other therapeutics.

In one embodiment, the antigen-presenting cells or the lymphocytes are administered systemically, e.g., by injection. Alternately, one can administer locally rather than systemically, for example, via injection directly into tissue. The pharmaceutical composition may be in a depot or sustained release formulation. Furthermore, one can administer in a targeted drug delivery system, for example, in a liposome that is coated with tissue-specific antibody. The liposomes can be targeted to and taken up selectively by the tissue.

Pharmaceutical compositions may be administered directly, endoscopically, intratracheally, intratumorally, intravenously, intralesionally, intramuscularly, intraperitoneally, regionally, percutaneously, topically, intrarterially, intravesically, or subcutaneously. Compositions may be administered 1, 2, 3, 4, 5, 6, 7, 8, 9, 10, 11, 12, 13, 14, 15, 16, 17, 18, 19, 20 or more times, and they may be administered every 1, 2, 3, 4, 5, 6, 7, 8, 9, 10, 11, 12, 13, 14, 15, 16, 17, 18, 19, 20, 21, 22, 23, 24 hours, or 1, 2, 3, 4, 5, 6, 7 days, or 1, 2, 3, 4, 5 weeks, or 1, 2, 3, 4, 5, 6, 7, 8, 9, 10, 11, 12 months.

### Combination Therapy

The pharmaceutical composition can be given subsequent to, preceding, or contemporaneously with other therapies including therapies that also elicit an immune response in the subject. For example, the subject may previously or concurrently be treated by chemotherapy, radiation therapy, surgery, other forms of immunotherapy, anti-angiogenic agents, anti-viral agents, and hormonal agents. Such other therapies preferably are provided in such a way so as not to interfere with the immunogenicity of the compositions of the present invention.

The pharmaceutically acceptable composition can be administered at any time that is appropriate. For example, the administering can be conducted before or during traditional therapy of a subject having a tumor burden, and continued after the tumor becomes clinically undetectable. The administration also can be continued in a subject showing signs of recurrence.

The chemotherapeutic agent may be naturally occurring or synthetic, for example as described in “Cancer Chemotherapeutic Agents”, American Chemical Society, 1995, W. O. Foye Ed. The chemotherapeutic agents may be compounds interacting with or binding tubulin, growth factor receptor antagonists, alkylating agents or platinum compounds, anthracyclines, as DNA intercalators or as DNA cross-linking agents, including DNA minor-groove binding compounds, anti-metabolites, bleomycin type antibiotics, inhibitors of DNA transcribing enzymes, and especially the topoisomerase I or topoisomerase II inhibitors, chromatin modifying agents, anti-mitotic agents, cell-cycle inhibitors, proteasome inhibitors, enzymes, hormones, hormone antagonists, hormone inhibitors, inhibitors of steroid biosynthesis, steroids, cytokines, hypoxia-selective cytotoxins, inhibitors of cytokines, lymphokines, antibodies directed against cytokines, oral and parenteral tolerance induction agents, supportive agents, chemical radiation sensitizers and protectors, photo-chemically activated drugs, synthetic poly- or oligonucleotides, optionally modified or conjugated, non-steroidal anti-inflammatory drugs, cytotoxic antibiotics, antibodies targeting the surface molecules of cancer cells, antibodies targeting growth factors or their receptors, inhibitors of metalloproteinases, metals, inhibitors of oncogenes, inhibitors of gene transcription or of RNA translation or protein expression, complexes of rare earth elements, and photo-chemotherapeutic agents.

Non-limiting examples of chemotherapeutic agents include paclitaxel (taxol), docetaxel, a vinca alkaloid such as navelbine, vinblastin, vincristin, vindesine or vinorelbine, an alkylating agent or a platinum compound such as melphalan, cyclophosphamide, an oxazaphosphorine, cisplatin, carboplatin, oxaliplatin, satraplatin, tetraplatin, iproplatin, mitomycin, streptozocin, carmustine (BCNU), lomustine (CCNU), busulfan, ifosfamide, streptozocin, thiotepa, chlorambucil, a nitrogen mustard such as mechlorethamine, an immunomodulatory drug such as thalidomide and its derivatives, or revimid (CC-5013)), an ethyleneimine compound, an alkylsulphonate, daunorubicin, doxorubicin (adriamycin), liposomal doxorubicin (doxil), epirubicin, idarubicin, mitoxantrone, amsacrine, dactinomycin, distamycin or a derivative thereof, netropsin, pibenzimol, mitomycin, CC-1065, a duocarmycin, mithramycin, chromomycin, olivomycin, a phtalanilide such as propamidine or stilbamidine, an anthramycin, an aziridine, a nitrosourea or a derivative thereof, a pyrimidine or purine analogue or antagonist or an inhibitor of the nucleoside diphosphate reductase such as cytarabine, 5-fluorouracile (5-FU), uracil mustard, fludarabine, gemcitabine, capecitabine, mercaptopurine, cladribine, thioguanine, methotrexate, pentostatin, hydroxyurea, or folic acid, an acridine or a derivative thereof, a rifamycin, an actinomycin, adramycin, a camptothecin such as irinotecan (camptosar) or topotecan, an amsacrine or analogue thereof, a tricyclic carboxamide, an histonedeacetylase inhibitor such as SAHA, MD-275, trichostatin A, CBHA, LAQ824, or valproic acid, a proteasome inhibitor such as bortezomib, a small molecule VEGF receptor antagonist such as vatalanib (PTK-787/ZK222584), SU-5416, SU-6668, SU-11248, SU-14813, AZD-6474, AZD-2171, CP-547632, CEP-7055, AG-013736, IM-842 or GW-786034, an antagonist of the mitogen-activated protein kinase such as BAY-43-9006 or BAY-57-9006, a dual EGFR/HER2 antagonist such as gefitinib, erlotinib, CI-1033 or GW-2016, an EGFR antagonist such as iressa (ZD-1839), tarceva (OSI-774), PKI-166, EKB-569, HKI-272 or herceptin, a quinazoline derivative such as 4-[(3-chloro-4-fluorophenyl)amino]-6-{[-4-(N,N-dimethylamino)-1-oxo-2-but-en-1-yl]amino}-7-((S)-tetrahydrofuran-3-yloxy)-quinazoline or 4-[(3-chloro-4-fluoro-phenyl)amino]-6-{[4-(homomorpholin-4-yl)-1-oxo-2-bu-ten-1-yl]amino}-7-[(S)-(tetrahydrofuran-3-yl)oxy]-quinazoline, or a pharmaceutically acceptable salt thereof, an inhibitor of the transcription factor complex ESX/DRIP130/Sur-2, an inhibitor of HER-2 expression, such as the heat shock protein HSP90 modulator geldanamycin and its derivative 17-allylaminogeldanamycin or 17-AAG, a protein kinase receptor antagonist which is not classified under the synthetic small molecules such as atrasentan, rituximab, cetuximab, Avastin™ (bevacizumab), IMC-1C11, erbitux (C-225), DC-101, EMD-72000, vitaxin, imatinib, and an antibody targeting the surface molecules of cancer cells such as apolizumab or 1D09C3.

### Cancer

The cancer that can be treated or prevented by the present methods and compositions includes, but is not limited to, solid malignancies and hematologic malignancies. The cancer may be primary or metastatic. The cancer may be Stage 0, Stage I, Stage II, Stage III, or Stage IV. The cancer also can be characterized as benign or malignant. The cancer may be metastatic, progressive and/or refractory. The cell that expresses a tumor-associated antigen can be any type of cell. The cell can be a cancer cell, a precancerous cell, or a cell-type predisposed to developing cancer.

The subject can either have a neoplastic disease (e.g., a tumor), or be at risk of developing the neoplastic disease. Subjects can be characterized by clinical criteria, for example, those with advanced neoplastic disease or high tumor burden exhibiting a clinically measurable tumor. A clinically measurable tumor is one that can be detected on the basis of tumor mass (e.g., by palpation, MRI, CAT scan, X-ray). Thus, for example, the pharmaceutically acceptable composition in accordance with the present invention can be administered to subjects with advanced disease with the objective of mitigating their condition. Preferably, a reduction in tumor mass occurs as a result of administering the pharmaceutically acceptable composition of the present invention, but any clinical improvement constitutes a benefit. Clinical improvement includes decreased risk or rate of progression or reduction in pathological consequences of a tumor, for example.

By way of another example, the subject can be one that has a history of cancer and has been responsive to another mode of therapy. The other therapy may have included e.g., surgical resection, radiotherapy, chemotherapy, and other modes of immunotherapy whereby as a result of the other therapy, the subject presents no clinically measurable tumor. However, the subject can be one determined to be at risk for recurrence or progression of the cancer, either near the original tumor site, or by metastases. Such subjects can be further categorized as high-risk and low-risk subjects. The subdivision can be made on the basis of features observed before or after the initial treatment. These features are known in the clinical arts, and are suitably defined for each different cancer. Features typical of high risk subgroups are those in which the tumor has invaded neighboring tissues, or which show involvement of lymph nodes. Thus, for example, a pharmaceutical composition of the present invention can be administered to the subject to elicit an anti-cancer response primarily as a prophylactic measure against recurrence. Preferably, administering the composition delays recurrence of the cancer, or more preferably, reduces the risk of recurrence (i.e., improves the cure rate). Such parameters can be determined in comparison with other patient populations and other modes of therapy.

Cancers that may be evaluated by methods and compositions of the invention include cancer of the bladder, blood, bone, bone marrow, brain, breast, colon, esophagus, gastrointestine, gum, head, kidney, liver, lung, nasopharynx, neck, ovary, pancreas, prostate, skin, stomach, testis, tongue, or uterus. In addition, the cancer may specifically be of the following histological type, though it is not limited to these: neoplasm, malignant; carcinoma; carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; androblastoma, malignant; sertoli cell carcinoma; leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malig melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

In one embodiment, the cancer treated or diagnosed by the present methods or compositions is multiple myeloma. In another embodiment, the cancer treated or diagnosed by the present methods or compositions is lymphoma. In a third embodiment, the cancer treated or diagnosed by the present methods or compositions is breast cancer. In a fourth embodiment, the cancer treated or diagnosed by the present methods or compositions is adenocarcinoma, which may be from breast, colon, liver, stomach, etc. In a fifth embodiment, the cancer treated or diagnosed by the present methods or compositions is metastatic solid malignancy which may or may not demonstrate a measurable response to first-line, conventional systemic therapy. In a sixth embodiment, the cancer treated or diagnosed by the present methods or compositions is progressive and/or refractory solid malignancy.

### Kits

The present invention further pertains to a kit containing the present pharmaceutical composition. The kit or container holds an effective amount of a pharmaceutical composition for carrying out the methods or producing the compositions described herein and/or instructions for producing or using the compositions for therapy of a patient or subject having or suspected of having or at risk of developing cancer.

The compositions of the present invention can be supplied in unit dosage or kit form. Kits can comprise various components of the pharmaceutically acceptable composition or vaccines thereof provided in separate containers as well as various other active ingredients or agents including chemotherapeutic agents.

Administering to both human and non-human vertebrates is contemplated within the scope of the present invention. Veterinary applications also are contemplated. Generally, the subject is any living organism in which an immune response can be elicited. Examples of subjects include, without limitation, humans, livestock, dogs, cats, mice, rats, and transgenic species thereof.

The following examples of specific aspects for carrying out the present invention are offered for illustrative purposes only, and are not intended to limit the scope of the present invention in any way.

### Examples

### Example 1

### Phase I/II Study of Low-Dose Cyclophosphamide, Tumor Associated Peptide Antigen-Pulsed Dendritic Cell Therapy and Low Dose Granulocyte-Macrophage Colony Stimulating Factor, as Consolidation Therapy in Patients with Metastatic Solid Malignancies, or in Patients with Progressive and/or Refractory Solid Malignancies

In this phase I/II study we will examine the feasibility, toxicity, immune response and antitumor activity of TAPA-pulsed DC therapy in patients with metastatic SM who may or may not demonstrate a measurable response to first-line, conventional systemic therapy, or in patients with progressive and/or refractory SM.

Selected TAPAs will be used to pulse DCs ex vivo, in the presence of a highly immunogenic maturation cocktail [9, 12, 18, 19]. Patients will also be treated with low-dose CYP prior to each DC vaccination, in an attempt to decrease the number and activity of Tregs. Low-doses of GM-CSF will be administered following each DC vaccination, in order to optimize immune responses in patients with relapsed/refractory SM. In a recent study conducted by others, dendritic cells loaded with tumor lysates have been combined with GM-CSF, pegylated IFN and cyclophosphamide to treat patients with refractory SM [9].

The regime to treat patients with metastatic SM (who may or may not demonstrate a tumor response to conventional first-line systemic therapy) or patients with relapsed and/or refractory SM, and whose tumor cells express at least one TAPA, include, using low-dose CYP followed by an autologous, monocyte-derived, TAPA-pulsed DC vaccine and low-dose GM-CSF. This treatment regime will result in TAPA-specific CD4+ T-cell and CD8+ CTL responses without significant toxicities. CD4+ T-cell and CD8+ CTL responses generated against specific TAPAs may translate into clinical antitumor activity.

The primary objective of Phase I is to determine the toxicity of low-dose CYP followed by TAPA-pulsed DC therapy and low-dose GM-CSF administration, in patients with metastatic SM, or in patients with progressive and/or refractory SM.

The secondary objectives of Phase II include (1) determining immune responses associated with low-dose CYP followed by TAPA-pulsed DC therapy and low-dose GM-CSF administration, in patients with metastatic SM, or in patients with progressive and/or refractory SM; and (2) determining the anti-cancer response associated with low-dose CYP followed by TAPA-pulsed DC therapy and low-dose GM-CSF administration in patients with metastatic SM, or in patients with progressive and/or refractory SM.

The methods disclosed herein may be used to treat or prevent solid malignancy or hematologic malignancy. In one embodiment, the malignancy is lymphoma. In another embodiment, the malignancy is multiple myeloma. In a third embodiment, the malignancy is breast cancer.

### Study Design

Patients diagnosed with metastatic solid malignancies (SM), who have responded to first-line conventional therapy, will be candidates for this Phase I/II study. Following confirmation of disease response to first-line systemic therapy, eligible patients will have their tumor cells analyzed for the expression of a specific panel of Tumor Associated Peptide Antigens (TAPAs), including Sp17, Ropporin, AKAP-4, PTTG1, Span-xb, Her-2/neu, HM1.24, NY-ESO-1 and MAGE-1. Patients whose tumors express one or more of these TAPAs will receive three (3) days of subcutaneous Granulocyte Macrophage Colony Stimulating Factor (GM-CSF) to increase bone marrow production of monocytes and dendritic cell (DC) precursors, and whole blood will be obtained by phlebotomy and/or leukapheresis performed for generation of autologous DCs. Patient's DCs will be generated, and activated by pulsing/loading them with the TAPA(s) relevant for each particular patient. Patients will receive 5 days of low-dose cyclophosphamide prior to each vaccination with TAPA-pulsed DCs to decrease Treg activity. TAPA-pulsed DCs will be administered at a dose of 1×10⁷ DCs at least two (2) days following cyclophosphamide administration. DC vaccination schedule will be once every 14 days via subcutaneous (SC) and intradermal (ID) injections for a total of 6 vaccinations. Low dose GM-CSF will also be administered SC for 5 consecutive days, starting six (6) hours after each TAPA-pulsed DC treatment, to optimize immune responses. Patients will be followed on a weekly basis (or more frequently if required) to evaluate treatment-related toxicity. Immune responses and anti-tumor responses will also be evaluated. Continuation and stopping rules for the study will be defined based on toxicity/tolerability (Phase I) and/or immune responses (Phase II).

Alternatively, patients diagnosed with progressive and/or refractory solid malignancies (SM), who have failed conventional therapy, will be candidates for this Phase I/II study. Following confirmation of disease progression and/or refractoriness, eligible patients will have their tumor cells analyzed for the expression of a specific panel of Tumor Associated Peptide Antigens (TAPAs), after which similar treatments as described above will follow.

The more specific protocol of the study is as follows. After patients are enrolled in the study program due to cancer cell expression of one or more of the relevant TAPAs by RT-PCR and/or Western blot, IHC, ELISA, they consent for either leukapheresis or phlebotomy. The leukapheresis or phlebotomy product is processed. The PBMCs are separated over Ficoll density gradient centrifugation. PBMC are pelleted and resuspended in CellGro DC serum free media (CellGenix, NH, USA) with L-glutamine. PBMCs are counted using a hemacytometer and viability determined using trypan blue (1:1) exclusion. For generation of a total DC vaccine bank all PBMCs are transferred to T150 flasks for monocyte sorting and iDC (immature DC) generation. For generation of only one fresh DC vaccine dose five/sixths (⅚^{th}) of the total number of PBMCs are cryopreserved in five (5) or more 2 ml NUNC vials. The remaining one-sixth (⅙^{th}) of the Ficoll-purified PBMC are utilized for generation of iDCs. PBMCs are washed and resuspended in T-150 tissue culture flasks at 1.0×10⁸ per flask in CellGro DC serum free media with L-glutamine and incubated at 37° C. for 2 hours in a 5% CO₂ incubator.

Following incubation, non-adherent cells are removed by three gentle washes with CellGro DC serum free media and adherent cells cultured in CellGro DC serum free media plus 10% autologous (patient) plasma, 800 U/ml of IL-4 and 1000 U/ml of GM-CSF, and incubated at 37° C. and 5% CO² for six (6) to eight (8) days (average seven (7) days). Fresh IL-4/GM-CSF are added on days two (2), four (4) and six (6). DC cultures are observed every other day. Morphology and level of confluence are noted. On days six (6) or seven (7) immature DCs will be counted to determine Total Volume Count (TVC). 1×10⁶ DCs with supernatant are removed for the gram stain, fungal, aerobic and anaerobic culture on days six (6) or seven (7) (prior to peptide loading/maturation cytokine cocktail pulsing). QC samples are removed after the cells have been washed and the TVC determined.

DCs are kept on T-150 flasks at a density of approximately 5×10⁵ DC cells/ml for peptide pulsing or alternatively, washed with phosphate buffered saline (PBS) and transferred (adherent and non-adherent DCs) to 50 ml conical tubes. DC culture is then pulsed with 20 μg/mL of one or more of the relevant tumor associated peptide antigens (TAPAs) (i.e., one or more peptides derived from Sp17, AKAP-4, Ropporin, PTTG-1, HMI.24, Her-2/neu, NY-ESO-1, MAGE-1, SPAN-Xb; see Table 1) for 4 hours followed by the addition of DC maturation cocktail (IL-1β and TNFα at 50 ng/ml, INF α at 1000 IU/ml and poly (I:C) at 20 μg/ml). DC culture is then incubated at 37° C. and 5% CO² for 16-24 hrs (average 20 hrs). After 16-24 hrs of incubation, pulsing/maturation treatment is stopped by centrifugation and peptide/cytokine containing medium removed. DCs are washed twice and resuspend DCs in D-PBS 1× (Gibco). Number of pulsed/mature DCs is determined (based on DC phenotype release assay results) and the appropriate dose loaded into a syringe. For patients for whom a total DC vaccine bank is generated and cryopreserved, DC vials containing the appropriate number of DCs will be thawed, DC viability determined and contents transferred into one or more syringe(s) with a 23 gauge needle and sterile saline solution for a total volume of 1 cc. The DC vaccine release criteria include: a passing result for the phenotype release assay is defined as cells expressing >70% CD86, CD80, CD83, CD58, CD1a, HLA-DR and <10% CD14, within the DC gate, DC viability of more than 80%, and negative test results for endotoxin, mycoplasma, fungal, aerobic and anaerobic cultures.

### Study Population

A minimum of six (6) subjects may be enrolled and receive DC vaccination to evaluate toxicity of the proposed vaccination strategy. Up to seventeen (17) patients may be enrolled for determination of immunological efficacy, assuming no overt toxicity is encountered during the safety evaluation.

### Inclusion Criteria

Patients are at least eighteen (18) years of age with histologically proven metastatic SM and whose SM demonstrates a response to conventional, first-line systemic therapy. Alternatively, patients can be at least eighteen (18) years of age with histologically proven, progressive and/or refractory SM.

Patients must express one or more of the following TAPAs; Sp17, AKAP-4, Ropporin, PTTG-1, Span-xb, Her-2/neu, HM1.24, NY-ESO-1 and MAGE-1, by either RT-PCR and/or immunocytochemistry, Western blotting or ELISA, in neoplastic cells.

Patients must not have any active infectious process; must have a negative test for HIV, Hepatitis A, B, and C; must not be receiving active immunosuppressive therapy; must have discontinued systemic antineoplastic therapy (including systemic corticosteroids) at least 4 weeks prior to enrollment; may not have any known allergy to GM-CSF; must be willing to provide at least 250-500 mls of whole blood obtained by phlebotomy and/or consent to leukapheresis for DC generation.

Inclusion criteria also include presence of measurable or evaluable disease; adequate renal and hepatic function (creatinine<2.0 mg/dl, bilirubin<2.0 mg/dl, AST and ALT<4× upper limit of normal range); adequate hematologic function (Platelets>60,000/mm³, lymphocytes>1,000 mm³, neutrophils>750/mm³, hemoglobin>8.5 g/dl); Karnofsky performance status >70%; eExpected survival greater than 6 months.

### Exclusion Criteria

Exclusion criteria include patients without confirmed metastatic SM and/or response to conventional, first-line systemic therapy (alternatively, patients without confirmed relapsed or refractory SM); patients without measurable or evaluable disease; patients receiving cytotoxic therapy, radiation therapy, immunotherapy or non-topical steroids, within 4 weeks of enrollment; active immunosuppressive or cytotoxic therapy (excluding topical steroids) for any other condition; persistent fever (>24 hours) documented by repeated measurement or active, uncontrolled infection within 4 weeks of enrollment; active ischemic heart disease or history of myocardial infarction within six months; active autoimmune disease, including, but not limited to, Systemic Lupus Erythematosus (SLE), Multiple Sclerosis (MS), Ankylosing Spondylitis (AS), and Rheumatoid Arthritis (RA); active second invasive malignancy, other than basal cell carcinoma of the skin; life expectancy of less than 6 months; patients with contraindications to GM-CSF; patients who have received organ transplantations.

### Study Schedule

Pre-treatment evaluations include completion of systemic therapy and confirmation of response to conventional first-line systemic therapy (alternatively, completion of systemic therapy and/or confirmation of progressive and/or relapsed SM); CBC, differential leukocyte counts, and baseline biochemical and/or radiographic evaluation of disease status no more than 28 days before the first (1^{st}) DC vaccination; completed baseline delayed-type hypersensitivity response (DTH) skin tests with specific TAPA(s) between two (2) and seven (7) days prior to 1st DC vaccination; infectious disease panel, as required for safe handling of blood/leukapheresis product (includes HIV, hepatitis A, B, C, and any mandated viral screens), no more than 14 days before phlebotomy/leukapheresis; GM-CSF at a dose of 250 mcg/day, subcutaneously, for three consecutive days immediately prior to phlebotomy/leukapheresis; phlebotomy/leukapheresis within 2 weeks of enrollment and 4 weeks prior to beginning DC vaccination schedule; manufacture of DC vaccine bank; baseline Immune Assays; initiation of treatment with low-dose CYP (day −7 to −3) and DC vaccination schedule (day 0).

### Safety Endpoints

The primary endpoints for this Phase I/II trial are safety and efficacy. The trial will be halted if Dose Limiting Toxicity (DLT, defined below) occurs in 2 or more of 6 patients receiving DC vaccination. If DLTs are observed in no more than 1/6 patients during the Phase I/toxicity phase, the treatment will advance to the Phase II/efficacy level. Immune response will be evaluated in all patients.

### Dose-Limiting Toxicity (DLT)

1. Grade III or higher allergic reaction. Grade III is defined as symptomatic bronchospasm requiring medication, edema or angioedema, and Grade IV is defined as anaphylaxis.
2. Grade II or higher autoimmune reaction. Grade II is defined as an autoimmune reaction involving a non-essential organ or function requiring treatment.
3. Grade III or higher hematologic or non-hematologic toxicity including fever (>40° C. for >24 hours).
4. Grade III injection site reaction (severe or prolonged ulceration or necrosis).
5. Toxicity will be graded according to the National Cancer Institute Common Terminology Criteria for Adverse Events (NCI-CTCAE), version 4.0.
6. If, at any time, a patient develops recurrent/progressive disease, defined as increasing clinical, radiologic, biochemical, or histological evidence of disease since entry into the study, he or she will be removed from the study.

### Immunological Efficacy Endpoint

The main secondary endpoint of this Phase I clinical study is immunological efficacy (IE), such as detection of a TAPA-specific immune response in SM patients receiving DC vaccination. Immune responses will be evaluated in all patients enrolled on the study. A minimum of 6 patients shall receive at least 3 DC vaccinations (evaluation at day 42) before an interim analysis of IE is performed. With regard to IE, the accrual goal will be seventeen (17) patients. Stopping criteria, with regard to IE, will be based on defined immune responses and will follow a Simon 2-stage design with a null hypothesis of 5% IE (p₀=0.05) and alternative hypothesis of 35% IE (p₁=0.35) with desired significance level (α) and desired power (1-β) of 0.05 and 0.9, respectively. Interim analysis of immune response will be conducted after accrual of 6 subjects, following completion of 42 days. If no immune responses are observed at interim analysis (0/6 patients), early stopping will be considered based on lack of efficacy of the intervention. With this rule, the probability of early termination (if the alternative hypothesis is true) is seven and one half percent (7.5%). If three (3) or more patients, out of a total of seventeen (17) develop TAPA-specific immune responses, then the null hypothesis of 5% IE can be rejected and results will be deemed favorable to move to phase II. Immune response rates will be compared among dose levels upon completion of the trial.

### Immune Response Criteria

In this study we will determine immune response (IR) based on the Distribution Free Resampling (DFR) method proposed and described by Moodie et at [66]. This non-parametric statistical method allows for control of the overall false positive rate if multiple antigens are considered and avoids parametric assumptions about the data set that may be false.

The IFN-γ ELISpot assay has been used extensively for determining immune responses in clinical studies evaluating cancer vaccines. The magnitude of IFN-γ ELISpot responses induced by cancer vaccines has been suggested to correlate with disease outcome and survival.

Immune Efficacy Endpoint will be determined as follows: (a) Positive T-cell cytokine IFN-γ, TNF-α and/or IL-17 ELISpot assay for ex vivo T cell cytokine expression following stimulation with relevant TAPA. For each patient sample, the IFN-γ ELISpot assay (and/or TNF-α or IL-17) will be performed in triplicate using a minimum of three wells per replicate, as described by the manufacturer. A concurrent negative control will be assayed in triplicate using a minimum of six wells per replicate. In addition, experimental replicates with large variance ratios (variance of replicates divided by median+1) will be excluded and re-assayed, if possible. A minimum of 5 spots will be considered the detection threshold for the IFN-γ ELISpot. The DFR method utilized will employ a null hypothesis of less or equal to a two-fold increase between negative control background and experimental means and the data set will be analyzed using the runDFR Web Tool. P values of <0.05 will be considered significant for an IR; and (b) positive DTH skin tests with relevant TAPA (DTH skin tests will be performed at the site of prior DC vaccination, if possible).

Determination of IE will follow a Simon 2-stage design and patients will be evaluable for an immune response after the third (3^{rd}) vaccination.

### Enzyme-Linked Immunosorbent Spot (ELISpot)

Upon challenge, the CD8+ cytotoxic T lymphocytes (CTL) recognize specific tumor-associated (e.g., Sperm protein 17, Her-2/neu, HM1.24, NYESO-1, MAGE-1, SPAN-Xb) in conjunction with MHC class I molecules, leading to secretion of interferon-gamma (IFN-γ) or other cytokines and lysis of cells expressing the specific TAPA. In addition, the CD4+ T helper lymphocytes recognize antigenic peptides in conjunction with MHC class II molecules, also leading to the secretion of IFN-γ which in turn affects other aspects of the immune response.

The number of antigen-specific precursor T cells available at the time of challenge will determine the magnitude of the immune-response and may ultimately affect the course of immune response by IFN-γ.

An ELISpot assay capable of detecting IFN-γ gamma-producing precursor T cells in a sample of peripheral blood mononuclear cells (PBMC) can be utilized to estimate the precursor frequency. The PBMC are serially diluted and placed in microplate wells coated with anti-human IFN-γ antibody. They are cultured with the specific TAPA for 20 hours, resulting in the re-stimulation of the precursor cells and secretion of IFN-γ or other cytokines of interest. The cells are washed away, leaving the secreted IFN-γ bound to the antibody-coated wells in concentrated areas where the cells were sitting. The captured IFN-γ is detected with biotinylated anti-human IFN-γ antibody followed by an alkaline phosphatase-conjugated anti-biotin antibody. The addition of insoluble alkaline phosphatase substrate results in dark spots in the wells at the sites where the cells were located, leaving one spot for each T cell that secreted IFN-γ. The number of spots per well is directly related to the precursor frequency of antigen-specific T cells.

Immune-Related Response Criteria (irRC)*

Baseline tumor assessment is determined by the sum of the products of the two largest perpendicular diameters (SPD) of all index lesions (five lesions per organ, up to 10 visceral lesions and five cutaneous index lesions) is calculated.

At each subsequent tumor assessment, the SPD of the index lesions and of new, measurable lesions (≧5×5 mm; up to 5 new lesions per organ) are added together to provide the total tumor burden:

Tumor Burden=SPD(index)lesions+SPD(new)lesions

Time-point response assessment using irRC. Percentage changes in tumor burden per assessment time point describe the size and growth kinetics of both conventional and new, measurable lesions as they appear.

At each tumor assessment, the response in index and new, measurable lesions is defined based on the change in tumor burden (after ruling out irPD). Decreases in tumor burden must be assessed relative to baseline measurements (i.e., the SPD of all index lesions at screening).

Overall Response Using the irRC:

The overall response according to the irRC is derived from time-point response assessments (based on tumor burden) as follows:
irCR: complete disappearance of all lesions (whether measurable or not, and no new lesions) confirmation by a repeat, consecutive assessment no less than 4 wk from the date first documented
irPR: decrease in tumor burden ≧50% relative to baseline confirmed by a consecutive assessment at least 4 wk after first documentation
irSD: not meeting criteria for irCR or irPR, in absence of irPD
irPD: increase in tumor burden ≧25% relative to nadir (minimum recorded tumor burden) confirmation by a repeat, consecutive assessment no less than 4 wk from the date first documented
   Derivation of irRC Overall Responses:

| Measurable Response | | | |
|---|---|---|---|
| Index and new, measurable | Nonmeasurable (NM) Response | | Overall Response |
| lesions (tumor burden), * % | Non-index | New, NM lesions | Definition |
| ↓100 | Absent | Absent | irCR† |
| ↓100 | Stable | Any | irPR† |
| ↓100 | Unequivocal progression | Any | irPR† |
| ↓≧50 | Absent/Stable | Any | irPR† |
| ↓≧50 | Unequivocal progression | Any | irPR† |
| ↓<50 to <25↑ | Absent/Stable | Any | irSD |
| ↓<50 to <25↑ | Unequivocal progression | Any | irSD |
| ≧25? | Any | Any | irPD† |

* Decreases assessed relative to baseline, including measurable lesions only (>5 × 5 mm).
†Assuming response (irCR) and progression (irPD) are confirmed by a second, consecutive assessment at least 4 wk apart.

Patients are considered to have irPR or irSD even if new lesions were present, as long as they met the respective thresholds of response as described above.

Patients are not considered to have irPD if new lesions were present and the tumor burden of all lesions did not increase by ≧25%.

In contrast to irCR, irPR, and irPD, a response of irSD does not require confirmation. It is important to note that irCR, irPR, and irSD include all patients with CR, PR, or SD by WHO criteria as well as those patients that shift to these irRC categories from WHO PD.

Patients with irSD, particularly those with slow-declining tumor burden ≧25% from baseline at the last tumor assessment, are considered clinically meaningful because they show an objectively measurable reduction in tumor burden without reaching the 50% threshold that defines irPR.

If a patient is classified as having irPD at a post-baseline tumor assessment, then confirmation of irPD by a second scan in the absence of rapid clinical deterioration is required. The definition of confirmation of progression represents an increase in tumor burden ≧25% compared with the nadir at two consecutive time points at least 4 wk apart. It is recommended that this be done at the discretion of the investigator because follow-up with observation alone may not be appropriate for patients with a rapid decline in performance status.

Confirmation of irPD allows for the capture of all observed responses using the irRC, as most of these late responding patients have a trend toward response within 4 wk after initial irPD. Whereas WHO criteria consider any new measurable lesion to indicate PD, determination of immune-related best overall response (irBOR) is based on changes in total tumor burden from the baseline (nadir, for irPD) tumor assessment, regardless of any initial increase in baseline lesions or the appearance of new lesions.

* Wolchok et al. Guidelines for the Evaluation of Immune Therapy Activity in Solid Tumors: Immune-Related Response Criteria. Clin Cancer Res 15(23):7412-20, 2009

Reasons for discontinuation of study include: high frequency of limiting toxicities (no safe dose determined, with starting dose determined to be above MTD); and lack of immune response.

DC vaccination strategies have been studied clinically in many different diseases. Both monocyte-derived DCs and CD34+-derived DCs have been used in the presence of serum-free mediums, autologous serum-containing mediums, or fetal calf serum-containing mediums. Because these cells have been generated from autologous cells, their administration either intravenously (IV), subcutaneously (SC) or intradermally (ID) has not been associated with any significant adverse effects. Minor adverse effects may include low grade fever and local reactions, such as erythema, at the sites of injection.

In a recently conducted phase I clinical trial of DC vaccination of patients with early-stage cervical cancer (BB-IND 11307), no adverse side effects were observed or reported by subjects following immunization beyond the immediate discomfort associated with injection [10]. The patients were monitored during treatment with complete blood counts and serum chemistries that included liver and renal function tests and electrolytes. No alterations in liver and renal function were detected. The safety of DC vaccination was also recently demonstrated in a study combining autologous DC vaccine against the melanoma antigen MART-1 and the cytotoxic T lymphocyte associated antigen 4 (CTL-4) antagonist tremelimumab [67]. The only toxicity reported in this study was related to grade I-II pruritic skin eruptions previously associated with CTLA4 antagonists. No toxicity attributed to DC vaccination alone was reported. One of the major concerns regarding DC vaccination with self-tumor antigens is the possible induction of autoimmunity. Vitiligo has been seen in some melanoma patients, but no cases of severe autoimmune reactions have been reported.

### Screening Visits and Procedures

Screening evaluations will include the following: History and physical examination; Karnofsky performance status; CBC and differential leukocyte count; and tests for hepatic function and renal function. Diagnostic studies, including biopsies, appropriate imaging studies, and serum and/or urine tumor marker measurements (if indicated), will be obtained within four (4) weeks prior to enrollment. Confirmation of expression of one (1) or more TAPA by neoplastic cells, by, e.g., RT-PCR, immunohistochemistry, ELISA or Western blotting, prior to enrollment.

TABLE 2
Karnofsky Performance Status Scale Definitions Rating (%) Criteria

| | |
|---|---|
| 100 | Normal no complaints; no evidence of |
| | disease. |
| 90 | Able to carry on normal activity; Minor signs or symptoms |
| | of disease. |
| 80 | Normal activity with efforts; some signs or |
| | symptoms of disease. |
| 70 | Cares for self; unable to carry on normal |
| | home and care for most activity or to do active work. |
| 60 | Requires occasional assistance, but is able |
| | to care for most of his personal needs. |
| 50 | Requires considerable assistance and |
| | frequent medical care. |
| 40 | Disabled; requires special care and assistance. |
| 30 | Severely disabled; hospital admission is |
| | indicated although death not imminent. |
| 20 | Very sick; hospital admission necessary; |
| | Active supportive treatment necessary. |
| 10 | Moribund; fatal processes progressing |
| | rapidly. |
| 0 | Dead |

Peripheral blood mononuclear cells will be harvested by phlebotomy and/or leukapheresis, via peripheral or central venous access, after 3 days of GM-CSF priming.

Phlebotomy/leukapheresis will be conducted within two (2) weeks of enrollment and four (4) weeks of the first DC vaccination.

Oral cyclophosphamide (CYP) treatment (100 mg/day) will begin seven (7) days prior to each TAPA-pulsed DC vaccination. Patients will receive five (5) days of CYP treatment.

TAPA-pulsed DC will be administered at a starting vaccine dose of 1×10⁷ DC in injection-grade saline solution containing heat-inactivated autologous serum. The vaccine volume will be up to 1.0 ml and will be administered subcutaneously (SC) and intradermally (ID) in the inguinal or axillary folds in order to increase proximity to local lymph node draining basins and optimize propagation of TAPA-pulsed DCs to secondary lymphoid organs. A maximum of 0.5 ml will be injected in a single site, both SC and ID (total volume 1.0 ml). The same site(s) will be used for repeated vaccinations unless a grade 2 or greater injection site reaction occurs, in which case a new site in the inguinal or axillary fold will be selected. Six (6) DC vaccines will be administered at 14 day intervals, plus or minus 3 days, to maximize patient convenience and protocol adherence. Patients will be observed for up to six (6) hours following each vaccine dose administration.

### Vaccination Schedule:

| Day 0 | Day 14 | Day 28 | Day 42 | Day 56 | Day 70 |
|---|---|---|---|---|---|
| Vaccine 1 | Vaccine 2 | Vaccine 3 | Vaccine 4 | Vaccine 5 | Vaccine 6 |

GM-CSF will be administered at a dose of 50 μg/day for five (5) consecutive days beginning six (6) hours after each TAPA-pulsed DC vaccination.

Blood for immune assays (up to 100 ml) will be drawn 8-10 days before the 1st DC vaccination, at the time of the 2nd, 3rd, 4^{th}, 5^{th} and 6^{th} DC vaccinations, plus at 14 and 60 days (plus or minus 10 days) after the 6^{th} DC vaccination.

DTH skin tests to assess cellular immune responses against relevant TAPAs will be conducted between 8-10 days before the 1st DC vaccination, at the time of the 3^{rd} and 6^{th} DC vaccinations, plus 14 and 60 days (plus or minus 10 days) after the 6^{th} DC vaccination.

### Final Study Visit

The final study visit will take place approximately 60 days (plus or minus 10 days) after the last (6^{th}) DC vaccination. Procedures will include a blood draw and DTH skin tests for responsiveness to TAPAs.

Laboratory tests at the final study visit will include hepatic and renal profile, CBC, and differential leukocyte counts.

Appropriate imaging studies will be also obtained.

### Post-Study Follow-Up

If study patients show no evidence of recurrent or progressive disease, additional DC vaccinations may be administered at 3 month intervals. Otherwise, patients will be followed indefinitely and treated per standard of care by the study investigator and/or patient's primary physician(s).

Blood for immune assays (up to 100 ml) will be drawn 14 and 60 days (plus or minus 10 days) after the 6^{th} DC vaccination.

### Study Materials and Procedures

### TAPA-Pulsed Dendritic Cells (DCs)

DCs will be derived from monocyte precursors present in peripheral blood mononuclear cells (PBMC) cultures following phlebotomy and/or leukapheresis. Monocyte precursors will be cultured in CellGro serum free media (CellGenix, USA), 10% plasma from patients or human AB serum (Biowhittaker) tested for endotoxin, 800 U/ml of IL-4 and 1000 U/ml of GM-CSF (CellGenix, USA) for seven (7) days. DC vaccine will be prepared by “pulsing” immature DCs with relevant, recombinant TAPAs (20 μg/ml) for four (4) hours followed by the addition of a DC maturation cytokine cocktail containing IL-10 and TNFα at 50 ng/ml (CellGenix, USA), poly (I:C) at 20 μg/ml (Hemispherx or InvivoGene, USA) and INFα at 1000 IU/ml (Humanzyme, USA) for an additional 16 to 24 hours. Successful generation of mature DCs will be confirmed in each case by immunophenotyping for classical DC markers, including CD1a, CD11c, CD40, CD80, CD83, CD86 and HLA-DR. Recombinant, clinical grade TAPAs will be obtained from CS Bio Company, Inc., Menlo Park, Calif. Following the maturation period, and after extensive washing to remove any unbound protein, DCs will be aliquoted into six or more equal volumes for patient administration or cryopreservation until further. Alternatively, DCs for the first vaccine dose will be generated from one sixth (⅙^{th}) of the original pool of PBMCs, with subsequent DC vaccine doses generated from cryopreserved PBMCs prior to vaccination day.

Mature DCs (or PBMCs for later generation of fresh mature DCs) will be cryopreserved and stored in liquid nitrogen until use.

A minimum of 6 and a maximum of 12 vials of TAPA-pulsed DCs (containing no less than 1×10⁷ DCs each) will be cryopreserved as a master vaccine bank for each patient. TAPA-pulsed DC vaccine will be frozen in DC medium plus 90% heat-inactivated autologous plasma (or AB human serum) and 10% dimethyl sulfoxide. Alternatively, ⅚^{th} of the original pool of PBMCs will be cryopreserved, as described above, for subsequent thawing and generation of fresh DCs prior to each vaccination schedule. This process may improve the viability of DCs.

### Generation of Tumor Associated Peptide Antigen-Pulsed Dendritic Cells (DCs)

Isolation of Monocytes from Peripheral Blood Mononuclear Cells (PBMC)

For the specimens which arrive as a phlebotomy or leukopack product from a leukapheresis, the cells are manually separated over Ficoll-HyPaque density gradient centrifugation.

PBMC are pelleted and resuspended in CellGro DC serum free media (CellGenix, NH, USA) with L-glutamine.

The PBMCs are counted with a hemacytometer and viability determined using trypan blue 1:1. DCs are generated either as a total vaccine bank prior to patient administration or individually prior to each administration. For generation of a total DC vaccine bank, all PBMCs isolated from phlebotomy and/or leukapheresis are processed and the final DC vaccine product cryopreserved until use. For generation of individual DC vaccine doses, excess PBMCs that will not be used for the generation of the first vaccine dose, are cryopreserved in 5 or more 2 ml NUNC vials. The remaining Ficoll-purified PBMC will be utilized for generation of fresh DCs, for the first DC vaccine injection. The subsequent five injections/doses of DCs will be prepared from frozen PBMC.

Generation of iDCs (Immature DCs) from Monocytes

PBMCs are harvested by Ficoll Hypaque density gradient centrifugation. The PBMCs are washed and resuspended in DC-medium and transferred in T-150 tissue culture flasks in CellGro DC serum free media (CellGenix, NH, USA) with L-glutamine. The cells are incubated at 37° C. for 2 to 4 hours in a 5% CO₂ incubator. Following incubation, non-adherent cells are removed by one to four gentle washes with DPBS and adherent cells are cultured in CellGro DC medium plus 1% to 10% autologous (patient) plasma or 1% to 10% heat inactivated normal human AB serum, 800 U/ml of GM-CSF and 1000 U/ml of IL-4, and incubated at 37° C. and 5% CO₂ for 4 to 7 days. Fresh IL-4/GM-CSF are added on days two (2), four (4), and six (6). Human AB serum (Sigma-Aldrich, USA) will be used only when patients' autologous plasma is unavailable.

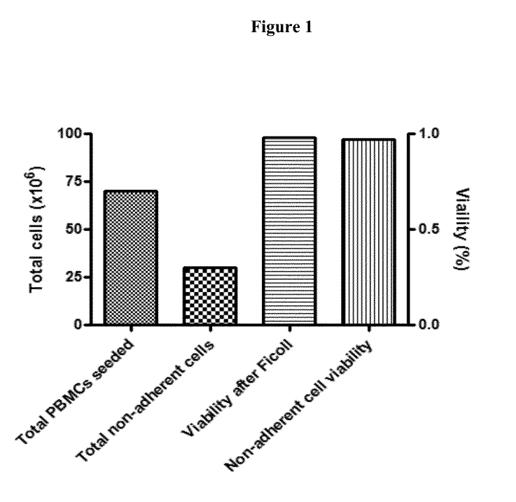

FIG. 1 shows a representative result obtained after Ficoll-purification of 35 mL of peripheral blood obtained from a healthy individual. 70×10⁶ cells were left to adhere in a T-150 flask as indicated above (DC medium+1% autologous plasma) for 3 hours in a cell culture incubator. Viability after Ficoll purification was 98%. The non-adherent cells were collected and frozen in 90% normal human AB serum+10% DMSO to be used in the cytotoxicity assay. The adherent fraction represented about 40% of the total amount of PBMCs isolated. The blood may be from a subject having solid malignancy or hematologic malignancy. In one embodiment, the malignancy is lymphoma. In another embodiment, the malignancy is multiple myeloma. In a third embodiment, the malignancy is breast cancer.

On days four (4), five (5), or six (6), immature DCs are counted to determine Total Volume Count (TVC).

0.5×10⁶ DCs with supernatant are removed for gram stain, aerobic, anaerobic and fungal culture, mycoplasma testing and mycoplasma testing on days four (4), five (5), or six (6) (prior to peptide loading/maturation cytokine cocktail pulsing). QC samples will be removed after the cells have been washed and the TVC determined. Remove a 100 μl aliquot for cell counting on the hemacytometer with Trypan blue staining.

Harvest of iDCs

Wash non-adherent iDCs from each flask by gently flushing the flask with the cell suspension 5 to 10 times, then transfer the cell suspension to a 50 mL tube. Take an aliquot for trypan-blue counting.

Keep iDC at a density of approximately 2 to 10×10⁶ cells/ml. If necessary, concentrate the cells by centrifuging at 100 to 300×g for 5 to 10 minutes at 10-20° C. and discard the excess medium before resuspending the pellet.

### Maturation of DC and Peptide Pulsing

Keep iDC at a density of approximately 2 to 10×10⁶ cells/ml. If necessary, concentrate the cells by centrifuging at 100 to 300×g for 5 to 10 minutes at 10-20° C. and discard the excess medium before resuspending the pellet.

Transfer the cell suspension in a conical 50 mL or 15 mL tube and add the maturation cytokine cocktail, then transfer the cell suspension to a new flask (up to 50×10⁶ cells in a 75 cm² growth area).

The maturation cytokine cocktail contains IL-1β and TNFα at 50 ng/ml, INF-α at 1,000 U/ml and poly (I:C) at 20 μg/ml. DC culture is then incubated at 37° C. and 5% CO₂ for 16 to 72 hours.

DC culture is then harvested (the adherent cells, if present, will be harvested by washing with PBS and by the use of a cell scraper), and re-adjusted at a density of 2 to 10×10⁶ cells/mL.

DCs are pulsed with 20 μg/mL of one or more of the relevant tumor associated peptide antigens (TAPAs) (i.e., peptides derived from Sp17, AKAP-4, Ropporin, PTTG-1, HMI.24, Her-2/neu, NY-ESO-1, MAGE-1, and/or SPAN-Xb. See Table 1 for the peptide sequences) for two (2) to four (4) hours. Working stock for the above TAPAs is 1 to 10 mg/ml, depending on the solubility of each TAPA.

After two (2) to four (4) hours of incubation, the pulsing is stopped by centrifuging the DC in the tubes at 100 to 300×g for 5 to 10 minutes at 10-20° C. and eliminating the supernatant. If using multi-wells, transfer the DC suspension in 15 or 14-mL conical tubes before centrifuging. Pulsed DC are then resuspended in 1 to 10 mL with fresh DC-medium 1% to 10% autologous (patient) plasma or 1% to 10% heat-inactivated normal human AB serum. Following pulsing and after viability count (trypan-blue), 0.9×10⁶ DCs are collected to perform flow-cytometry quality control analysis, and 1.5×10⁶ DCs with medium to perform sterility QC tests.

As an alternative, the pulsing with TAPAs can be performed prior to exposure to the cytokine maturation cocktail. iDCs are pulsed and subsequently matured, as follows. Keep iDCs (derived from the GM-CSF/IL-4 culture) at a density of approximately 2 to 10×10⁶ cells/ml. If necessary, concentrate the cells by centrifuging at 100 to 300×g for 5 to 10 minutes at 10-20° C. and discard the excess medium before resuspending the pellet.

Then, DC is pulsed as described above. After two (2) to four (4) hours of incubation, the pulsing is stopped as described above. Pulsed DC are then resuspended at 2 to 10×10⁶ cells/mL in CellGro DC medium plus 1% to 10% autologous (patient) plasma or 1% to 10% heat inactivated normal human AB serum, 800 U/ml of GM-CSF and 1000 U/ml of IL-4. Then, the maturation cocktail is added as described above and incubated at 37° C. and 5% CO₂ for 16 to 72 hours. Matured DC are then centrifuged at 100 to 300×g for 5 to 10 minutes at 10-20° C., the supernatant is discarded and the DC are resuspended in 1 to 10 mL with fresh DC-medium 1% to 10% autologous (patient) plasma or 1% to 10% heat inactivated normal human AB serum. Following pulsing and after viability count (trypan-blue), 0.9×10⁶ DCs are collected to perform flow-cytometry quality control analysis, and 1.5×10⁶ DC with medium to perform sterility QC test.

### Harvest of Pulsed/Mature DCs

Calculate the proportion of DCs based on flow cytometry (see DC Phenotype Release Assay) and determine if the culture contains the appropriate number/dose of DCs for the entire treatment (approximately up to 1-6×10⁷ DCs depending on the generation of one dose vs total vaccine bank).

After washing the DC with 2 volumes of PBS, resuspend the DC pellet in PBS at 10⁶ to 10⁷/mL in PBS and load the appropriate number of DCs into a syringe with a 23 gauge needle for a total volume of 1 cc.

### DC Phenotype Release Assay and Cell Viability

An aliquot from the harvested, pulsed mDC culture is stained and the flow-cytometry analysis performed. These are performed from mature/pulsed DC on days 5, 6, 7, 8, 9, or 10 from the beginning of the iDC differentiation, depending on the day when the maturation is performed.

A passing result for the phenotype release assay is defined as cells expressing >70% CD86, CD80, CD83, CD58, HLA-DR and <10% CD14, within the DC gate.

Viability for fresh cells are determined by Trypan blue exclusion. A viability of more than 80% is required for release.

The DC population is understood to be larger and more internally granular than the lymphocyte population. Therefore the DC population lies above and over from the lymphocyte population in a FS/SS scattergram.

The release assay has two sections; the first determines the percentage of live cells that are DCs, and the second determines the percentage of DCs that are positive for certain cell surface markers.

Following 5 days of culture with GM-CSF and IL-4, non-adherent, immature DC (20×10⁶ were harvested as described above and concentrated to 2×10⁶/mL in the same medium. Then, the maturation cocktail was added, and the DC suspension was transferred to a T-75 flask and incubated for 24 hours in 5% CO₂ at 37° C. Then, suspension and adherent cells were collected and pulsed with TAPAs as described above, for 2 hours in 14-mL polypropylene tubes (2 mL/tube with 2×10⁶ cells/tube). Then, an aliquot of 0.4×10⁶ cells was removed for flow-cytometry quality control.

The following staining is performed: Isotype control FITC, Isotype control PE, CD86 (FITC), CD58 (PE), HLA-DR (FITC), CD83 (PE), CD14 (FITC), and CD80 (PE). Cells will be stained according to standard protocol. Approximately 0.9×10⁶ cells will be required for the assay.

Draw one bitmap around the entire DC population. Draw a second bitmap around the entire lymphocyte population. For the DC only bitmap perform a separate single color analysis for CD86, CD83, CD80, CD58, CD1a, HLA-DR and CD14. For the lymphocyte only bitmap perform a single color analysis for CD86, CD83, CD80, CD58, CD1a, HLA-DR and CD14.

### Determining Percent DCs

From the isotype control tube, the percent DC is the percentage of cells within the DC only bitmap, as opposed to all of the cells in the FS/SS scattergram. The % DC is used in various sections of the DC process to determine the total number of DC in culture.

### Analysis of Cell Surface Markers for Release Assay

From the DC bitmap only, acceptance criteria are: CD86 greater than or equal to 70% positive; CD80 greater than or equal to 70% positive; CD83 greater than or equal to 70% positive; CD58 greater than or equal to 70% positive; HLA-DR greater than or equal to 70% positive; and CD14 less than or equal to 10% positive.

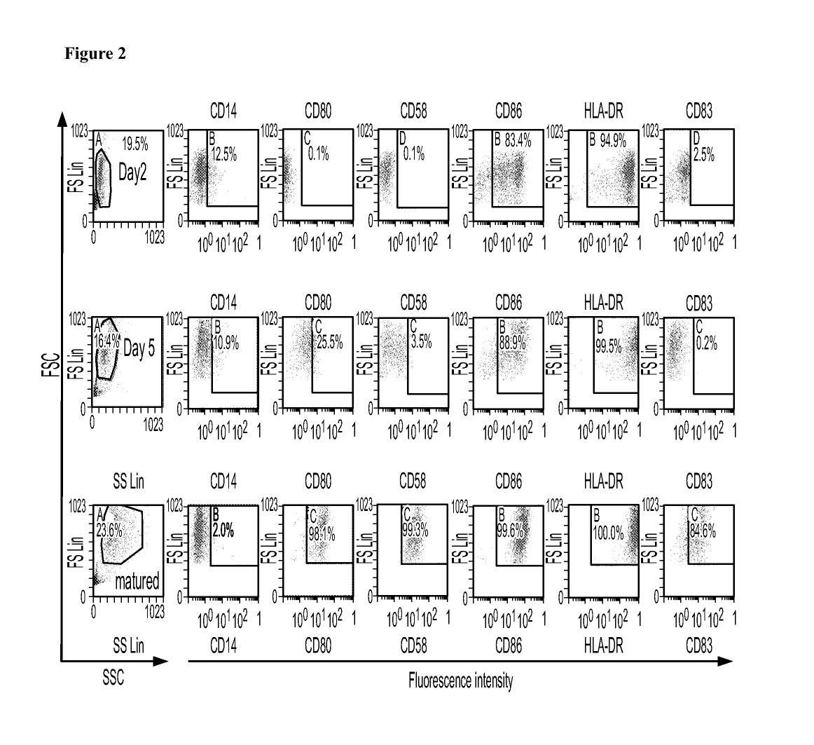

FIG. 2 shows representative phenotypic characterization of DCs after 2 or 5 days of GM-CSF stimulation, and after 20 μg/mL SP17(103-111) peptide (SEQ ID NO. 1) pulse. It is evident that the DC population increased in dimensions, as depicted by the FSC/FSC dot-plot, and that the maturation successfully induced the expected up-regulation of the maturation markers, CD80, CD83, CD86, CD58, and HLA-DR, while reduced the expression of the monocyte marker, CD14.

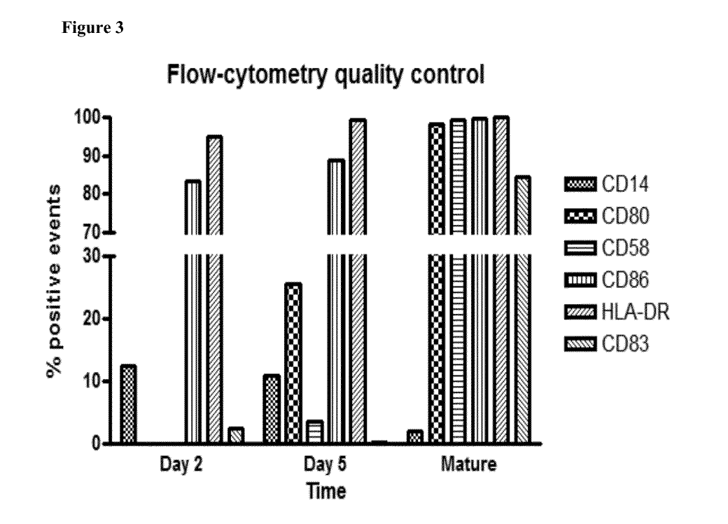

FIG. 3 shows results of the effects of GM-CSF and IL-4 and of the maturation/pulsing stimulation on the phenotype of monocyte-derived DC.

QC Sampling after Harvest of Pulsed/Mature DCs

These are performed from mature/pulsed DC on days 5, 6, 7, 8, 9, or 10 from the beginning of the iDC differentiation, depending on the day when the maturation is performed.

### Endotoxin Test:

0.5×10⁶ cells with supernatant are removed for the endotoxin Limulus Amoebolysate (LAL) testing. The LAL test is performed using the QCL-1000 kit by the chromogenic method. A passing result of less than or equal to 1.0 IU/mL of treatment aliquot tested is required for release of the fresh DCs and administration to patients.

### Mycoplasma Test:

0.5×10⁶ cells with supernatant are removed for the mycoplasma assay. A mycoplasma culture is done. A 96 hour DNA fluorochrome results (Hoechst) is optional and is not required for administration of the fresh DC infusion. If performed, a passing result for the 96 hour Hoechst assay is “negative.”

### Sterility Test:

0.5×10⁶ cells with supernatant are removed for the fungal, aerobic and anaerobic bacterial culture, sensitivity and stat gram stain. Samples are observed on a continuous basis for 14 days. A negative gram stain on the day of harvest and negative culture at 24 hours (removed prior to peptide pulsing) is required for release of the initial fresh DC culture. A passing result for sterility testing is “negative” for the presence of microbial contamination in fungal and aerobic and anaerobic bacterial canisters.

### Final Record

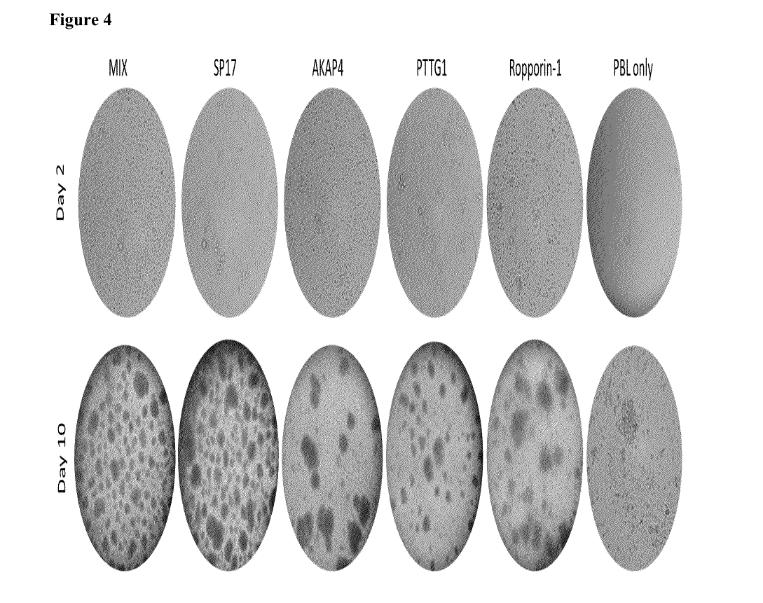

To evaluate the ability of matured/pulsed DC to induce specific autologous cytotoxic lymphocytes (CTL), an in vitro cytotoxicity was performed using the autologous pulsed dendritic cells as targets and in vitro generated CTL as effectors. To obtain CTL, 0.5×10⁶ mature/pulsed DC were mixed with 5×10⁶ autologous non-adherent PBMCs (PBL) obtained after the adhesion of monocytes (described above) on day 0. Such co-cultures were maintained in DC medium+2% autologous plasma, and they were supplemented with 10 μg/mL corresponding peptide and 10 U/mL IL-2. Every 2 days, 50% of medium was replaced with fresh medium, and fresh peptides and IL-2 were added. After 10 days, cells were counted and incubated with target cells at different ratios (from 40 to 20:1). As a negative control, CTL generated with DC pulsed with different TAPAs were incubated with autologous DC pulsed with the irrelevant antigen, E6 (HPV E6 protein). In the co-cultures, but not when PBL were cultured alone (without DC), we detected the formation of rosetta-like structure, which indicate interaction between T cells and DC (FIG. 4).

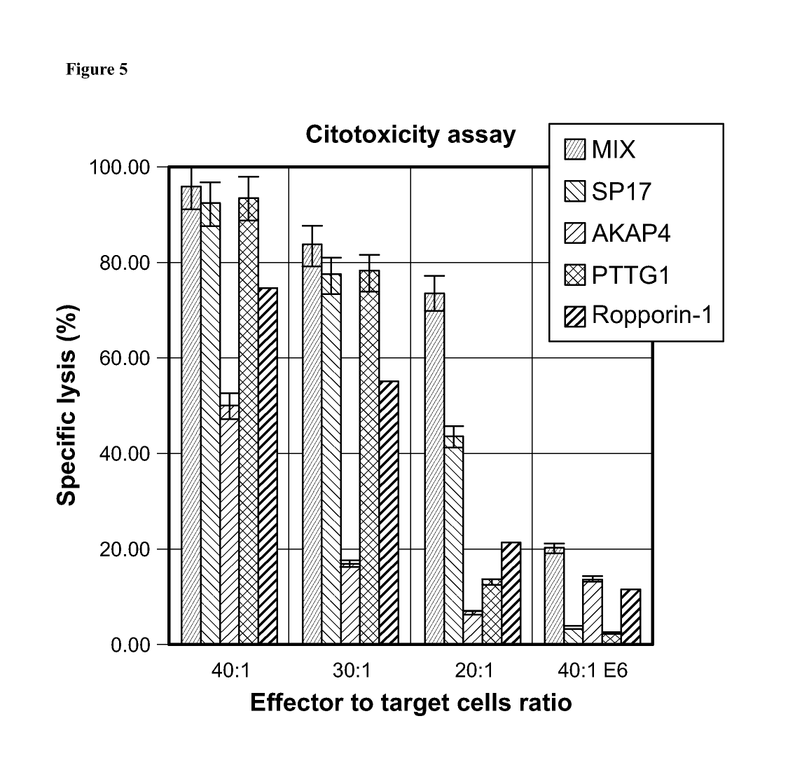

FIG. 5 shows representative cytotoxicity assay results obtained with DC pulsed with peptides derived from SP17, AKAP4, PTTG1, Ropporin-1 (see Table 1 for sequences), or a mix of these four antigens. The non-radioactive LDH cytotoxicity assay (Promega) was used to evaluate the specific lysis of TAPA-presenting DC. Results demonstrate that the DC generated with the protocol described here successfully induced the maturation and activation of antigen-specific cytotoxic lymphocytes. Lack of significant lysis of targets expressing the irrelevant antigen, E6, confirmed target specificity of the DC-primed CTL.

### Cryopreservation of Mature DCs

For cryopreservation of DCs, when a total DC vaccine bank is generated, centrifuge cells at 100 to 300×g for 5 to 10 minutes at 10-20° C., then resuspend in 5 to 10 mL of cold freezing medium (90% normal human AB serum+10% DMSO).

### Thawing of Frozen PBMC for DC Generation

Frozen PBMCs are thawed in the 37° C. water bath for 2 to 5 minutes. The product is then diluted in 9 volumes of DC medium (pre-warmed at 37° C.) supplemented with 2% autologous plasma, centrifuged at 100 to 300×g for 5 to 10 minutes at 10-20° C., and then transferred to the appropriate number of T-150 flasks.

### Thawing of Frozen Mature DCs for Patient Administration

Frozen mature DCs is thawed in the 37° C. water for 2 to 5 minutes. The product is then diluted in 9 volumes of DC medium (pre-warmed at 37° C.) supplemented with 2% autologous plasma Diluted in DC-medium and counted by Trypan blue exclusion method. A DC viability of more than 80% is required for release of vaccine dose. The product is then centrifuged at 100 to 300×g for 5 to 10 minutes at 10-20° C., resuspended in sterile PBS at the concentration of up to 10⁷ cells/mL, then transferred to one or more syringe(s) with a 23 gauge needle, each one to a volume of 1 ml for patient administration.

### Treatment

### Cyclophosphamide (CYP)

Patients will be treated with CYP orally at a dose of 100 mg/day for 5 days, beginning seven (7) days prior to each TAPA-pulsed DC vaccine dose (day −7 though day −3, days 7-11, days 21-25, days 35-39, days 49-53, days 63-67 corresponding to 6 treatments).

### Selection of TAPA-Pulsed DC Dose Levels

A phase I dose escalation clinical trial of DC vaccination in patients with cervical cancer indicated optimal stimulation of tumor antigen-specific cytotoxic T cell responses with a dose of 1.0×10⁷ DCs, in injection-grade saline containing 30% heat-inactivated autologous serum, and delivered SC and ID at 21 day intervals [10]. Thus, in this study we will explore one (1) dose of TAPA-pulsed DC vaccination (1×10⁷ DCs) and determine the toxicity, immune efficacy (IE) and clinical response in patients with progressive and/or refractory SM.

Preliminary studies indicate that leukapheresis (10 liter volume) followed by CliniMACS isolation of CD14+ cells and high density DC culture in G-Rex flasks provided an optimal yield of 2.5×10⁸ mature DCs from 10⁹ CD14+ cells. This would be sufficient for cryopreservation of 12 vials at 1×10⁷ DC/vial. Phlebotomy of 250 ml of whole blood yields approximately 1.2×10⁸ monocytes and 3×10⁷ mature DCs.

### TAPA-Pulsed DC Vaccination

If a total vaccine bank is generated, TAPA-pulsed DCs will be thawed out, washed once with sterile saline and resuspended in up to 1 ml injection-grade saline containing 10% autologous human serum. The vaccine volume will be up to 1.0 ml and half the volume (0.5 ml) will be administered SC and ID in the patient's inguinal or axillary fold, in order to increase proximity to local lymph node draining basins and optimize access of the TAPA-pulsed DCs to secondary lymphoid organs and propagation of the immune response. A maximum of 1.0 ml will be injected in a single site. Approximately half the volume per injection (0.5 ml) will be delivered SC and half ID, on a single site. The same site(s) will be used for repeated vaccinations unless a grade 2 or greater injection site reaction occurs, in which case a new site in the inguinal fold will be selected. Six DC vaccines will be administered at 14 day intervals, plus or minus 3 days, to maximize patient convenience and protocol adherence. Alternatively, TAPA-pulsed DCs will be generated prior to each vaccination and administered to patients every two (2) weeks, as planned. Patients will be observed for up to six (6) hours following each vaccine dose administration.

This is a phase I/II safety and efficacy study. The first six (6) patients will receive 1×10⁷ DCs divided in a subcutaneous (SC) and intradermal (ID) administration, every fourteen (14) days, for up to a maximum of six (6) treatments. The SC and ID DC vaccinations will be administered in normal saline with a total volume of 0.5 ml per injection (total vaccination dose-volume 1.0 ml). Prior to receiving the DC vaccination, all patients will receive premedication with diphenhydramine (50 mg) intravenously and acetaminophen (1000 mg) orally.

If one (1) or less than six (6) patients develops DLT, the Phase II/efficacy level will proceed. If two or more (≧2) of the first six (6) patients develop DLTs, the study will be terminated. A minimum of six (6) patients will be treated for evaluation of safety/toxicity (Phase I level). A maximum of seventeen (17) patients will be treated in the study for evaluation of immune efficacy and clinical response (Phase II level).

### GM-CSF Treatment

Following each DC vaccination, each patient will receive SC injections of low dose GM-CSF (50 mcg) daily for five (5) consecutive days, beginning six hours after the DC administration (days 0-4, days 14-18, days 28-32, days 42-46, days 56-60, days 70-74 corresponding to 6 treatments).

### Study Drug Quality and Safety Measures: TAPA-Pulsed Dendritic Cells (DCs)

TAPA-pulsed DCs will be administered SC and ID at 14 day intervals plus or minus 3 days. Safety tests before cryopreservation or release of TAPA-pulsed DCs including, Agar sterility test; mycoplasma test by approved kit; and/or endotoxin test by an approved independent testing laboratory. Endotoxin must be less than 1 IU/ml by the LAL method. Safety tests before administration of TAPA-pulsed DCs including, Gram stain prior to administration; cell viability by trypan blue exclusion; and/or broth culture sterility test.

Immunosuppressive or anti-inflammatory drugs (including hydrocortisone) that inhibit cellular immune responses should not be taken, unless otherwise indicated for the management of study-related toxicities or adverse events.

### Sample Size for Safety Evaluation

An initial cohort of six (6) subjects will receive low-dose CYP, TAPA-pulsed DC vaccination and low-dose GM-CSF for determination of safety (phase I level). Depending on the incidence of limiting toxicity at day 28, additional patients will receive treatment (up to seventeen (17)) for evaluation of efficacy (Phase II level).

### Sample Size for Evaluation of Immunological Efficacy

The accrual goal, with regard to efficacy, will be at least 17 patients. Stopping criteria for the study, based on Immune Efficacy (IE), will follow the efficacy rules described herein.

### Statistical Methods

For the immunological efficacy (IE) part of the study, a Simon 2-stage design with a null hypothesis of 5% IE (p₀=0.05) and alternative hypothesis of 35% IE (p₁=0.35), with desired significance level (α) and desired power (1-13) of 0.05 and 0.9, respectively, will be utilized. An interim analysis of immune response will be conducted after accrual and treatment of the first six (6) patients, following completion of 42 days (prior to 4^{th} DC vaccination). If no immune responses are observed at interim analysis, the study will be stopped based on lack of efficacy. The probability of early termination, if no immune responses are observed in the first six (6) patients and the alternative hypothesis is true, is 7.5%. If 3 or more responses out of a total of 17 patients are observed, the null hypothesis can be rejected and DC vaccination may move to a formal Phase II/III developmental stage.

### REFERENCES

1. Siegel R, Naishadman D, Jemal A. Cancer Statistics, CA Cancer J Clin 62:10-29, 2012.
2. Mellman I, Coukos G, Dranoff G. Cancer immunotherapy comes of age. Nature 480:480-489, 2011.
3. Banchereau J, Briere F, Caux C, et al. Immunobiology of dendritic cells. Ann Rev Immunol, 18(1), 767-811, 2000.
4. Figdor C G, De Vries I J M, Lesterhuis W J, et al. Dendritic cell immunotherapy: mapping the way. Nat Med, 10(5), 475-480, 2004.
5. Banchereau J and Palucka A K. Dendritic cells as therapeutic vaccines against cancer. Nat Rev Immunology, 5(4), 296-306, 2005.
6. Kalinski P, Muthuswamy R and Urban J. Dendritic cells in cancer immunotherapy: vaccines and combination immunotherapies. Expert Rev. Vaccines 12:285-295, 2013.
7. Scholm J. Therapeutic cancer vaccines: Current status and moving forward. J. Natl. Cancer Inst. 104:599-613, 2012.
8. Reyes D, Salazar L, Espinoza, et al. Tumour cell lysates-loaded dendritic cell vaccine induces biochemical and memory immune response in castration-resistant prostate cancer patients. Br. J. Cancer 109:1488-1497, 2013.
9. Alfaro C, Perez-Gracia J, Suarez N, et al. Pilot clinical trial of type 1 dendritic cells loaded with autologous tumor lysates combined with GM-CSF, pegylated IFN, and cyclophosphamide for metastatic cancer patients. J Immunol 187: 6130-6142, 2011.
10. Santin A, Bellone S, Palmieri M, et al. Human Papillomavirus Type 16 and 18 E7-Pulsed Dendritic Cell Vaccination of Stage IB or IIA Cervical Cancer Patients: a Phase I Escalating-Dose Trial. J Virol 82(4): 1968-1979, 2008.
11. Baek S, Kim C S, Kim S B, et al. Combination therapy of renal cell carcinoma or breast cancer patients with dendritic cell vaccine and IL-2: results from a phase I/II trial. J Transl Med 9:178, 2011.
12. Phuphanih S, Wheeler C, Rudnick J D, et al. Phase I trial of multiepitope-pulsed dendritic cell vaccine for patients with newly diagnosed glioblastoma. Cancer Immunol Immunother DOI 10.1007/s00262-012-139-0, 2012.
13. Akiyama Y, Ashita C, Kume A, et al. Alpha-type-1 polarized dendritic cell-based vaccination in recurrent high-grade glioma: a phase I clinical trial. DOI: 10.1186/1471-2407-12-623, 2012.
14. Bender, A., Sapp, M., Schuler, G., et al. Improved methods for the generation of dendritic cells from nonproliferating progenitors in human blood. J Immunolo Meth, 196(2), 121-135, 1996.
15. Timmerman M D, J M Levy, M D R. Dendritic cell vaccines for cancer immunotherapy. Ann Rev Med, 50(1), 507-529, 1999.
16. Ridgway D. The first 1000 dendritic cell vaccinees. Cancer Invest, 21(6), 873-886, 2003.
17. Chiriva-Internati M, Wang Z, Salati E, et al. Sperm protein 17 (Sp17) is a suitable target for immunotherapy of multiple myeloma. Blood, 100(3), 961-965, 2002.
18. Mailliard R B, Wankowicz-Kalinska A, Cai Q, et al. α-Type-1 Polarized Dendritic Cells A Novel Immunization Tool with Optimized CTL-inducing Activity. Cancer Res, 64(17), 5934-5937, 2004.
19. Bae W K, Umeyama A, Chung I J, et al. Uncarinic acid C plus IFN-γ generates monocyte-derived dendritic cells and induces a potent Th1 polarization with capacity to migrate. Cell Immunol, 266(1), 104-110, 2010.
20. Yang D H, Kim M H, Lee Y K, et al. Successful cross-presentation of allogeneic myeloma cells by autologous alpha-type 1-polarized dendritic cells as an effective tumor antigen in myeloma patients with matched monoclonal immunoglobulins. Ann Hematol, 90(12), 1419-1426, 2011.
21. Gerosa F, Baldani-Guerra B, Nisii C, et al. Reciprocal activating interaction between natural killer cells and dendritic cells. J Exp Med, 195(3), 327-333, 2002.
22. Nefedova Y, Nagaraj S, Rosenbauer A. et al. Regulation of dendritic cell differentiation and antitumor immune response in cancer by pharmacologic-selective inhibition of the janus-activated kinase 2/signal transducers and activators of transcription 3 pathway. Cancer Res, 65(20), 9525-9535, 2005.
23. Curiel T J. Regulatory T cells and treatment of cancer. Curr Op Immunol, 20(2), 241-246, 2008.
24. Frasca L, Fedele G, Deaglio S, et al. CD38 orchestrates migration, survival, and Th1 immune response of human mature dendritic cells. Blood, 107(6), 2392-2399, 2006.
25. Lea I A, Adoyo P, O'Rand M G Autoimmunogenicity of the human sperm protein Sp17 in vasectomized men and identification of linear B cell epitopes. Fertil. Steril. 67: 355-361, 1997.
26. Lim S H, Wang, Z, Chiriva-Internati M, et al. Sperm protein 17 as a novel cancer-testis (CT) antigen in Hematologic malignancies. Blood, 97: 1508-1510, 2001.
27. Chiriva-Internati M, Wang Z, Pochopien S, et al. Identification of a sperm protein 17 CTL epitope restricted by HLA-A1. Int J Cancer 107:863-865, 2003.
28. Chiriva-Internati M, Wang Z, Salati E, et al. Tumor vaccine for ovarian carcinoma targeting sperm protein 17. Cancer, 94(9), 2447-2453. 32, 2002.
29. Chiriva-Internat M, Mirandola L, Figueroa, J A, et al. Selective expression and immunogenicity of cancer/testis antigens SP17, AKAP4 and PTTG1 in non-small cell lung cancer: new candidates for active immunotherapy. Submitted: Chest, 2013
30. Dadabayeb A R, Wang Z, Zhang Y, et al. Cancer Immunotherapy Targeting Sp17: When should the laboratory findings be translated to the clinics? Am J Hematol 80:6-11, 2005.
31. Pawson, T. & Scott, J. D. Signaling through scaffold, anchoring and adaptor proteins. Science, 278:2075-2080, 1997.
32. Turner R M, Musse M P, Mandal A, et al. Molecular genetic analysis of two human sperm fibrous sheath proteins, AKAP4 and AKAP3, in men with dysplasia of the fibrous sheath. J Andro 22: 302-315, 2001.
33. Maurizio Chiriva-Internati, Raffaele Ferrari, Yuefei Yu, et al. AKAP-4: a novel cancer testis antigen for multiple myeloma. British J Haematol, 140, 465-468.
34. Mirandola L, Y Yu, Jenkins M et al. Tracking human multiple myeloma xenografts in NOD-Rag-1/IL-2 receptor gamma chain-null mice with the novel biomarker AKAP-4. BMC cancer 11: 394, 2011
35. Chiriva-Internati, M., Cobos, E. and Kast, W. M. Advances in immunotherapy of multiple myeloma: from the discovery of tumor associated antigens to clinical trials. International Reviews of Immunology, 2, 1-26, 2007.
36. Li Z, Meklaft F, Wang Z, et al. A yeast two-hybrid system using Sp17 identified Ropporin as a novel cancer-testis antigen in hematologic malignancies. Int. J. Cancer 121:507-1511, 2007.
37. Zhang X, Horwitz G A, Prezant T R, Valentini A, Nakashima M, Bronstein M D, Melmed S: Structure, Expression, and Function of Human Pituitary Tumor-Transforming Gene (PTTG). Molecular Endocrinology 13:156-166, 1999.
38. Wang Z, Lu Q Y, Chen P, Zhang P, Cong Y Q: Expression of pituitary tumor-transforming gene in patients with multiple myeloma. Zhongguo Shi Yan Xue Ye Xue Za Zhi 14:1143-1145, 2006.
39. Chiriva-Internati M, Ferrari R, Prabhakar M, et al. The pituitary tumor transforming gene 1 (PTTG-1): an immunological target for multiple myeloma.” J Transl Med 6: 15, 2008.
40. Wang Z, Zhang, Y, Liu H, et al. Gene expression and immunologic consequence of SPAN-Xb in myeloma and other hematologic malignancies. Blood, 101(3), 955-960, 2003.
41. Almanzar G, Olkhanud P V, Bodogai M, et al. Sperm-derived SPANX-B is a clinically relevant tumor antigen that is expressed in human tumors and readily recognized by human CD4+ and CD8+ T cells. Clin Cancer Res 15: 1954-1963, 2009.
42. Müller M R, Grünebach F, Kayser K, et al. Expression of Her-2/neu on Acute Lymphoblastic Leukemias Implications for the Development of Immunotherapeutic Approaches. Clin Cancer Res, 9(9), 3448-3453, 2003.
43. Potti A, Ganti A K, Koch M, et al. Immunohistochemical identification of Her-2/neu overexpression and DC117 (c-Kit) expression in multiple myeloma. Leuk Lymphoma 43:2427-2430, 2002.
44. Scardino A, Alves P, Gross D A, et al. Identification of Her-2/neu immunogenic epitopes presented by renal cell carcinoma and other epithelial tumors. Eur J Immunol 31:3261-3270, 2001.
45. Chiriva-Internati M, Liu Y, Weidanz J A, et al. Testing recombinant adeno-associated virus-gene loading of dendritic cells for generating potent cytotoxic T lymphocytes against a prototype self-antigen, multiple myeloma HM1. 24. Blood, 102(9), 3100-3107, 2003.
46. Walter-Yohrling J, Cao X, Callahan M, et al. Identification of genes expressed in malignant cells that promote invasion. Cancer Res 63:8939, 2003.
47. Ono K, Ohtomo T, Yoshida K, et al. The humanized anti-HM1. 24 antibody effectively kills multiple myeloma cells by human effector cell-mediated cytotoxicity. Mol Immunol, 36(6), 387-395, 1999.
48. Jager E, Gnjatic S, Nagata Y, et al. Induction of primary NY-ESO-1 immunity: CD8+ T lymphocyte and antibody responses in peptide-vaccinated patients with NY-ESO-1+ cancers. Proc Nat Acad Sci, 97(22), 12198-12203, 2000.
49. Jager E, Nagata Y, Gnjatic S, et al. Monitoring CD8 T cell responses to NY-ESO-1: correlation of humoral and cellular immune responses. Proc Nat Acad Sci, 97(9), 4760-4765, 2000.
50. Jungbluth A A, Ely S, DiLiberto M, et al. The cancer-testis antigens CT7 (MAGE-C1) and MAGE-A3/6 are commonly expressed in multiple myeloma and correlate with plasma-cell proliferation. Blood, 106(1), 167-174, 2005.
51. Traversari C, Van der Bruggen P, Luescher I, et al. A nonapeptide encoded by human gene MAGE-1 is recognized on HLA-A1 by cytolytic T lymphocytes directed against tumor antigen MZ2-E. J Exp Med 176:1453-1457, 1992.
52. Cheever M A and Higano C S. PROVENGE (Sipuleucel-T) in prostate cancer: the first FDA-approved therapeutic cancer vaccine. Clin Cancer Res, 17(11), 3520-3526, 2011.
53. Lutsiak M E, Semnani R T, De Pascalis R, Kashmiri S V, Schlom J, Sabzevari H. Inhibition of CD4+ CD 25+ T regulatory cell function implicated in enhanced immune response by low-dose cyclophosphamide. Blood 105:2862-8, 2005.
54. Greten T F, Ormandy L A, Fikuart A, H [symbol] ochst B, Henschen S, H [symbol] orningM, et al. Low-dose cyclophosphamide treatment impairs regulatory T cells and unmasks AFP-specific CD4b T-cell responses in patients with advanced HCC. J Immunother 33:211-8, 2010.
55. Ge Y, Domschke C, Stoiber N, Schott S, Heil J, Rom J, et al. Metronomic cyclophosphamide treatment in metastasized breast cancer patients: immunological effects and clinical outcome. Cancer Immunol Immunother 61:353-62, 2012.
56. Cerullo V, Diaconu I, Kangasniemi L, Rajecki M, Escutenaire S, Koski A, et al. Immunological effects of low-dose cyclophosphamide in cancer patients treated with oncolytic adenovirus. Mol Ther 19:1737-46, 2011.
57. Dung T. Le and Elizabeth M. Jaffee. Regulatory T-cell Modulation Using Cyclophosphamide in Vaccine Approaches: A Current Perspective. Cancer Res 72:3439-3444, 2012.
58. Kalantari T, Kamali-Sarvestani E, Ciric B, et al. Generation of immunogenic and tolerogenic clinical-grade dendritic cells. Immunol Res. 51: 153-160, 2011.
59. Kalinski P and Okada H. Polarized Dendritic Cells as Cancer Vaccines: Directing Effector type T Cells to Tumors. Semin Immunol. 2010 June; 22(3): 173-182.
60. van de Laar L, Coffer P J and Woltman A M. Regulation of dendritic cell development by GM-CSF: Molecular control and implications for immune homeostasis and therapy. Blood 119:3383-3393, 2013.
61. Dang Y, Wagner W, Gad E, et al. Dendritic cell-activating vaccine adjuvants differ in the ability to elicit antitumor immunity due to adjuvant-specific induction of immunosuppressive cells. Clin Cancer Res 18:1-10, 2012.
62. Clive K, Tyler J, Clifton G T, et al. Use of GM-CSF as an adjuvant with cancer vaccines: beneficial or detrimental? Expert Rev Vaccines 9:519-525, 2010.
63. Parmiani G, castelli C, Pilla L, et al. Opposite immune functions of GM-CSF administered as vaccine adjuvant in cancer patients. Ann Oncol 18:226-232, 2007.
64. Gjertsen M, Buanes T, Rosseland A, et al. Intradermal ras peptide vaccination with granulocyte macrophage colony stimulating factor as adjuvant: clinical and immunological responses in patients with pancreatic adenocarcinoma. Int J cancer 92:441-450, 2001.
65. Slingluff Jr C L, Petroni G R, Yamshchikov G V, et al. Clinical and immunologic results of a randomized phase II trial of vaccination using four melanoma peptides either administered in granulocyte-macrophage colony-stimulating factor in adjuvant or pulsed dendritic cells. J Clin Oncol, 21(21), 4016-4026, 2003.
66. Moodie Z, Price L, Gouttefangeas C, et al. Response definition criteria for ELISPOT assays revisited. Cancer Immunol Immunother 59:1489-1501, 2010.
67. Ribas A, Comi-Anduix B, Chmielowski B, et al. Dendritic cell vaccination combined with CTLCA4 blockade in patients with metastatic melanoma. Clin Cancer Res 15(19): 6267-76, 2009.
68. Wilke C M, Kryczek I, Wei S, et al. Th17 cells in cancer: help or hindrance? Carcinogenesis 32:643-649, 2011.

### Sequences:

| | | SEQ |
|---|---|---|
| Name | Sequence | ID NO. |
| Sp17(103-111) | ILDSSEEDK | 1 |
| AKAP-4(150-158) | YADQVNIDY | 2 |
| PTTG-1(70-78) | ATEKSVKTK | 3 |
| Ropporin(113-121) | FTEEIEWLK | 4 |
| Span-Xb(86-94) | DQMEEEEFI | 5 |
| Her-2/neu(106-114) | QLFEDNYAL | 6 |
| HM1.24(9-17) | FLVFVFVFL | 7 |
| NYESO-1(159-167) | LMWITQCFL | 8 |
| MAGE-1(248-256) | TLAKFSPYL | 9 |

What is claimed is:

1. A method of treating and/or preventing cancer in a subject, the method comprising the steps of:
(a) determining whether the subject expresses at least one tumor-associated antigen, wherein the tumor-associated antigen comprises Sp17, Ropporin, AKAP-4, PTTG1, Span-xb, Her-2/neu, HM1.24, NY-ESO-1, MAGE-1 or combinations thereof;
(b) loading antigen-presenting cells with at least one tumor-associated peptide antigen (TAPA) derived from at least one tumor-associated antigen expressed by the subject; and
(c) administering the antigen-presenting cells from step (b) to the subject.

2. The method of claim 1, wherein the TAPA comprises SEQ ID NOs: 1, 2, 3, 4, 5, 6, 7, 8, or combinations thereof.

3. The method of claim 1, wherein the TAPA is MHC class I-restricted.

4. The method of claim 3, wherein the MHC class I is HLA-A.

5. The method of claim 1, wherein the antigen-presenting cells are dendritic cells.

6. The method of claim 5, wherein the dendritic cells are derived from autologous monocytes.

7. The method of claim 6, wherein the monocytes are cultured in vitro to induce differentiation into dendritic cells.

8. The method of claim 7, wherein differentiation into dendritic cells is facilitated by a composition comprising IL-1β, TNFα, IFN-α and poly (I:C).

9. The method of claim 7, wherein the monocytes are isolated from the subject's blood.

10. The method of claim 9, wherein granulocyte-macrophage colony-stimulating factor (GM-CSF) is administered to the subject before the subject's blood is obtained.

11. The method of claim 1, wherein an immunosuppressive agent is administered to the subject before step (c).

12. The method of claim 11, wherein the immunosuppressive agent is an alkylating agent.

13. The method of claim 12, wherein the alkylating agent is cyclophosphamide.

14. The method of claim 1, wherein granulocyte-macrophage colony-stimulating factor (GM-CSF) is administered to the subject after step (c).

15. The method of claim 1, wherein the subject has solid malignancy or hematologic malignancy.

16. The method of claim 15, wherein the subject has solid malignancy which expresses at least one tumor-associated antigen.

17. The method of claim 15, wherein the subject has metastatic solid malignancy.

18. The method of claim 15, wherein the subject has progressive and/or refractory solid malignancy.

19. The method of claim 1, wherein in step (a) protein level and/or mRNA level of at least one tumor-associated antigen are assayed.

20. The method of claim 19, wherein the assay method comprises RT-PCR, Western blot, immunohistochemistry, enzyme-linked immunosorbent assay (ELISA), or combinations thereof.

21. A pharmaceutical composition comprising dendritic cells loaded with at least one tumor-associated peptide antigen (TAPA), wherein the TAPA comprises SEQ ID NOs: 1, 2, 3, 4, 5, 6, 7, 8, or combinations thereof.

22. A pharmaceutical composition comprising dendritic cells comprising nucleic acids encoding at least one tumor-associated antigen, wherein the tumor-associated antigen is Sp17, Ropporin, AKAP-4, PTTG1, Span-xb, Her-2/neu, HM1.24, NY-ESO-1, MAGE-1 or combinations thereof.
